SEQUENCE LISTING (38 sequences)

SEQ ID NO: 1 - protein, 6 aa, synthetic construct
  REGION 1..6: note = Synthetic: heavy chain variable region CDR1
SSYYMA

SEQ ID NO: 2 - protein, 5 aa, synthetic construct
  REGION 1..5: note = Synthetic: heavy chain variable region CDR1
DSYMS

SEQ ID NO: 3 - protein, 17 aa, synthetic construct
  REGION 1..17: note = Synthetic: heavy chain variable region CDR2
DMYPDNGDSSYNQKFRE

SEQ ID NO: 4 - protein, 8 aa, synthetic construct
  REGION 1..8: note = Synthetic: heavy chain variable region CDR3
APRWYFSV

SEQ ID NO: 5 - protein, 11 aa, synthetic construct
  REGION 1..11: note = Synthetic: light chain variable region CDR1
RASQDISNYLN

SEQ ID NO: 6 - protein, 7 aa, synthetic construct
  REGION 1..7: note = Synthetic: light chain variable region CDR2
YTSRLRS

SEQ ID NO: 7 - protein, 9 aa, synthetic construct
  REGION 1..9: note = Synthetic: light chain variable region CDR3
QQGHTLPPT

SEQ ID NO: 8 - protein, 117 aa, synthetic construct
  REGION 1..117: note = Synthetic: OX40 antibody VH sequence
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 9 - protein, 107 aa, synthetic construct
  REGION 1..107: note = Synthetic: OX40 antibody VL sequence
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 10 - protein, 5 aa, synthetic construct
  REGION 1..5: note = Synthetic: heavy chain variable region CDR1
NYLIE

SEQ ID NO: 11 - protein, 17 aa, synthetic construct
  REGION 1..17: note = Synthetic: heavy chain variable region CDR2
VINPGSGDTYYSEKFKG

SEQ ID NO: 12 - protein, 5 aa, synthetic construct
  REGION 1..5: note = Synthetic: heavy chain variable region CDR3
DRLDY

SEQ ID NO: 13 - protein, 11 aa, synthetic construct
  REGION 1..11: note = Synthetic: light chain variable region CDR1
HASQDISSYIV

SEQ ID NO: 14 - protein, 7 aa, synthetic construct
  REGION 1..7: note = Synthetic: light chain variable region CDR2
HGTNLED

SEQ ID NO: 15 - protein, 9 aa, synthetic construct
  REGION 1..9: note = Synthetic: light chain variable region CDR3
VHYAQFPYT

SEQ ID NO: 16 - protein, 114 aa, synthetic construct
  REGION 1..114: note = Synthetic: OX40 antibody VH sequence
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 17 - protein, 107 aa, synthetic construct
  REGION 1..107: note = Synthetic: OX40 antibody VL sequence
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 18 - protein, 215 aa, synthetic construct
  REGION 1..215: note = Synthetic: anti-CEA/anti-CD3 bispecific antibody
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, synthetic construct
  REGION 1..214: note = Synthetic: anti-CEA/anti-CD3 bispecific antibody
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 694 aa, synthetic construct
  REGION 1..694: note = Synthetic: anti-CEA/anti-CD3 bispecific antibody
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, synthetic construct
  REGION 1..451: note = Synthetic: anti-CEA/anti-CD3 bispecific antibody
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 453 aa, synthetic construct
  REGION 1..453: note = Synthetic: anti-CEA/anti-CD3 bispecific antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 23 - protein, 463 aa, synthetic construct
  REGION 1..463: note = Synthetic: anti-CEA/anti-CD3 bispecific antibody
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 24 - protein, 214 aa, synthetic construct
  REGION 1..214: note = Synthetic: anti-CEA/anti-CD3 bispecific antibody
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 213 aa, synthetic construct
  REGION 1..213: note = Synthetic: anti-CEA/anti-CD3 bispecific antibody
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 17 aa, synthetic construct
  REGION 1..17: note = Synthetic: heavy chain variable region CDR2
AIFTGSGAEYKAEWAKG

SEQ ID NO: 27 - protein, 13 aa, synthetic construct
  REGION 1..13: note = Synthetic: heavy chain variable region CDR3
DAGYDYPTHAMHY

SEQ ID NO: 28 - protein, 11 aa, synthetic construct
  REGION 1..11: note = Synthetic: light chain variable region CDR1
RASQGISSSLA

SEQ ID NO: 29 - protein, 7 aa, synthetic construct
  REGION 1..7: note = Synthetic: light chain variable region CDR2
GASETES

SEQ ID NO: 30 - protein, 12 aa, synthetic construct
  REGION 1..12: note = Synthetic: light chain variable region CDR3
QNTKVGSSYGNT

SEQ ID NO: 31 - protein, 123 aa, synthetic construct [sequence omitted]

SEQ ID NO: 32 - protein, 110 aa, synthetic construct [sequence omitted]

SEQ ID NO: 33 - DNA, 20 bp, synthetic construct
  misc_feature 1..20: note = Synthetic: PERK sgRNA 1
  source 1..20: mol_type = other DNA
agtcacggcgggcactcgcg

SEQ ID NO: 34 - DNA, 20 bp, synthetic construct
  misc_feature 1..20: note = Synthetic: PERK sgRNA 2
  source 1..20: mol_type = other DNA
tacggccgaagtgaccgtgg

SEQ ID NO: 35 - DNA, 20 bp, synthetic construct
  misc_feature 1..20: note = Synthetic: PERK sgRNA 3
  source 1..20: mol_type = other DNA
gcgtgactcatgttcgccag

SEQ ID NO: 36 - DNA, 19 bp, synthetic construct
  misc_feature 1..19: note = Synthetic: Luciferase sgRNA
  source 1..19: mol_type = other DNA
atcctgtccctagtggccc

SEQ ID NO: 37 - DNA, 20 bp, synthetic construct
  misc_feature 1..20: note = Synthetic: CHO-XBP1s Forward Primer
  source 1..20: mol_type = other DNA
ccttgtaattgagaaccagg

SEQ ID NO: 38 - DNA, 21 bp, synthetic construct
  misc_feature 1..21: note = Synthetic: CHO-XBP1s Reverse Primer
  source 1..21: mol_type = other DNA
ccaaaaggatatcagactcgg

(19) United States
(12) Patent Application Publication
(10) Pub. No.: US 2024/0190944 A1
(43) Pub. Date: Jun. 13, 2024

(54) MODIFIED CELLS FOR THE PRODUCTION OF A RECOMBINANT PRODUCT OF INTEREST

(71) Applicant: Genentech, Inc., South San Francisco, CA (US)

(72) Inventors: Shahram MISAGHI, South San Francisco, CA (US); Brian Megia Castellano, South San Francisco, CA (US); Danming Tang, South San Francisco, CA (US); Avi J. Ashkenazi, South San Francisco, CA (US); Bradley R. Snedecor, South San Francisco, CA (US)

(73) Assignee: Genentech, Inc., South San Francisco, CA (US)

(21) Appl. No.: 18/514,280

(22) Filed: Nov. 20, 2023

Related U.S. Application Data
(63) Continuation of application No. PCT/US22/30345, filed on May 20, 2022.
(60) Provisional application No. 63/191,781, filed on May 21, 2021.

(51) Int. Cl. C07K 16/00; C07K 14/47; C12N 9/12; C12N 15/90

(52) U.S. Cl. CPC C07K 16/00; C07K 14/4747; C12N 9/12; C12N 15/907; C12Y 207/11001; C07K 2317/14; C12N 2310/20

(57) ABSTRACT

The present disclosure relates to cells (e.g., Chinese Hamster Ovary (CHO) cells) that are modified to reduce or eliminate the expression of certain endogenous cellular proteins, and methods of using such cells in the production of a recombinant product of interest, e.g., a recombinant protein, a recombinant viral particle, or a recombinant viral vector. These modifications were specifically chosen to generate engineered cells with desired traits in several key areas, including improved cell culture performance (e.g., higher viability and product titers).

### CROSS-REFERENCE TO RELATED APPLICATIONS

This application claims priority to PCT Application No. PCT/US22/030345, filed May 20, 2022, which claims priority to U.S. Provisional Application No. 63/191,781, filed on May 21, 2021, the contents of which is incorporated by reference in its entirety.

### SEQUENCE LISTING

The instant application contains a Sequence Listing which has been submitted electronically in XML format and is hereby incorporated by reference in its entirety. Said sequence listing copy, created on Nov. 20, 2023, is named 00B206 1392SL.xml and is 52,769 bytes in size.

### 1. FIELD OF INVENTION

The present disclosure relates to cells (e.g., Chinese Hamster Ovary (CHO) cells) that are modified to reduce or eliminate the expression of certain endogenous proteins, and methods of using such cells in the production of a recombinant product of interest, e.g., a recombinant protein, a recombinant viral particle, or a recombinant viral vector. These modifications generate engineered cells with unexpectedly synergistic traits in key areas, including improved viability and higher product titers.

### 2. BACKGROUND

Due to the rapid advancement in cell biology and immunology, there has been an increasing demand to develop novel therapeutic recombinant products, e.g., recombinant proteins, recombinant viral particles, and recombinant viral vectors, for a variety of diseases including cancer, cardiovascular diseases and metabolic diseases. These biopharmaceutical candidates are commonly manufactured by commercial cell lines capable of expressing the products of interest. For example, CHO cells have been widely adapted to produce monoclonal antibodies.

Expression of certain proteins by cells are detrimental for cell culture performance. For example, proteins that promote apoptosis can decrease culture viability and productivity. In addition, in the course of biopharmaceutical production, recombinant product expression can impose a high proteostatic burden that elicits cellular adaptation. For example, cells often utilize the unfolded protein response (UPR), to mitigate increased proteostatic burdens. The UPR, however, can ultimately lead to decreased overall protein translation, negatively impacting the titer achieved for a recombinant product of interest.

Accordingly, there is a need in the art for more efficient methods, modified cells, and compositions for producing a recombinant product of interest, e.g., a recombinant protein, a recombinant viral particle, or recombinant viral vector, where the modified cells expressing the recombinant product of interest exhibit improved attributes relevant to cell viability and the titer associated with production of products of interest. Such improved cells can be achieved by modifying the genome of the cells (i.e., cell line engineering).

### 3. SUMMARY

In certain embodiments, the present disclosure is directed to a modified cell, wherein the cell is modified to reduce or eliminate the expression of two or more endogenous proteins relative to the expression of the endogenous proteins in an unmodified cell, wherein: (a) one or more of the endogenous proteins having reduced or eliminated expression promotes apoptosis of the modified cell during cell culture; and (b) one or more of the endogenous proteins having reduced or eliminated expression regulates the unfolded protein response (UPR).

In certain embodiments, the present disclosure is directed to a modified cell, wherein the cell is modified to reduce or eliminate the expression of two or more endogenous proteins relative to the expression of the endogenous proteins in an unmodified cell, wherein one or more endogenous proteins is selected from the endogenous protein group consisting of: BCL2 Associated X, Apoptosis Regulator (BAX); and BCL2 Antagonist/Killer 1 (BAK); and one of the endogenous proteins is Protein Kinase R-like ER Kinase (PERK).

In certain embodiments, the present disclosure is directed to a modified cell, wherein the expression of BAX, BAK, and PERK is reduced or eliminated.

In certain embodiments, the present disclosure is directed to the above described modified cell, where the modified cell is engineered to express a recombinant product of interest. In certain embodiments, the present disclosure is directed to the above described modified cell, where the modified cell is generated from a recombinant cell that expresses a recombinant product of interest. In certain embodiments, the present disclosure is directed to the above described modified cell, where the one or more endogenous proteins have no detectable expression. In certain embodiments, the present disclosure is directed to the above described modified cell, where the recombinant product of interest comprises a viral vector. In certain embodiments, the present disclosure is directed to the above described modified cell, where the recombinant product of interest comprises a viral particle. In certain embodiments, the present disclosure is directed to the above described modified cell, where the recombinant product of interest comprises a recombinant protein. In certain embodiments, the present disclosure is directed to the above described modified cell, where the recombinant protein is antibody or an antibody-fusion protein or an antigen-binding fragment thereof. In certain embodiments, the present disclosure is directed to the above described modified cell, where the antibody is a multispecific antibody or an antigen-binding fragment thereof. In certain embodiments, the present disclosure is directed to the above described modified cell, where the antibody consists of a single heavy chain sequence and a single light chain sequence or antigen-binding fragments thereof. In certain embodiments, the present disclosure is directed to the above described modified cell, where the antibody is a chimeric antibody, a human antibody or a humanized antibody. In certain embodiments, the present disclosure is directed to the above described modified cell, where the antibody is a monoclonal antibody. In certain embodiments, the present disclosure is directed to the above described modified cell, where the recombinant product of interest is encoded by an exogenous nucleic acid sequence integrated in the cellular genome at one or more targeted locations.

In certain embodiments, the present disclosure is directed to the above described modified cell, where the modified cell does not express detectable BAX, BAK, and PERK. In certain embodiments, the present disclosure is directed to the above described modified cell, where the modified cell expresses decreased levels of BAX, BAK, and PERK.

In certain embodiments, the present disclosure is directed to the above described modified cell, where the modified cell is a modified animal cell. In certain embodiments, the modified animal cell is a modified Sf9, CHO, HEK 293, HEK-293T, BHK, A549, or HeLa cell.

In certain embodiments, the present disclosure is directed to a composition comprising the above-described modified cells.

In certain embodiments, the present disclosure is directed to a method of producing a recombinant product of interest comprising: (a) culturing the above described modified cell; and (b) recovering the recombinant product of interest from a cultivation medium or the modified cells, wherein the modified cells expressing the recombinant product of interest exhibit reduced or eliminated expression of BAX, BAK, and PERK.

In certain embodiments, the present disclosure is directed to a method for producing a modified cell, comprising: (a) applying a nuclease-assisted and/or nucleic acid targeting BAX, BAK, and PERK, in the cell to reduce or eliminate the expression of said endogenous genes, and (b) selecting the modified cell wherein the expression of said endogenous genes have been reduced or eliminated as compared to an unmodified cell.

In certain embodiments of the above-described methods for producing a modified cell, the modification is performed before the introduction of the exogenous nucleic acid encoding the recombinant product of interest, or after the introduction of the exogenous nucleic acid encoding the recombinant product of interest. In certain embodiments, the nuclease-assisted gene targeting system is selected from the group consisting of CRISPR/Cas9, CRISPR/Cpf1, zinc-finger nuclease, TALEN or meganuclease. In certain embodiments, the reduction of gene expression is mediated by RNA silencing. In certain embodiments, the RNA silencing is selected from the group consisting of siRNA gene targeting and knock-down, shRNA gene targeting and knock-down, and miRNA gene targeting and knock-down.

In certain embodiments, the recombinant product of interest is encoded by a nucleic acid sequence. In certain embodiments, the nucleic acid sequence is integrated in the cellular genome of the modified cells at one or more targeted locations. In certain embodiments, the recombinant product of interest expressed by the modified cells is encoded by a nucleic acid sequence that is randomly integrated in the cellular genome of the modified cells.

In certain embodiments, the recombinant product of interest comprises a viral vector. In certain embodiments, recombinant product of interest comprises a viral particle. In certain embodiments, the recombinant product of interest comprises a recombinant protein. In certain embodiments, the recombinant protein is an antibody or an antibody-fusion protein or an antigen-binding fragment thereof. In certain embodiments, the antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody. In certain embodiments, the antibody is a monoclonal antibody.

In certain embodiments of the above-described methods comprise purifying the product of interest, harvesting the product of interest, and/or formulating the product of interest.

In certain embodiments of the above-described methods the modified cell is a modified animal cell. In certain embodiments, the modified animal cell is a modified Sf9, CHO, HEK 293, HEK 293T, BHK, A549, or HeLa cell.

In certain embodiments, the present disclosure is directed to modified cell that has a higher specific productivity than a corresponding isolated animal cell that comprises the polynucleotide and functional copies of each of the wild type Bax, Bak, and PERK genes.

In certain embodiments, the present disclosure is directed to modified cell that is more resistant to apoptosis than a corresponding isolated animal cell that comprises functional copies of each of the Bax, Bak, and PERK genes.

In certain embodiments, the present disclosure is directed to modified cell that is employed in fed-batch, perfusion, process intensified, semi-continuous perfusion, or continuous perfusion cell culture process.

### 4. BRIEF DESCRIPTION OF THE DRAWINGS

FIGS. 1A-1G. PDGFRa is downregulated by UPR activation. FIG. 1A and FIG. 1B depict that PDGFRa protein levels and mRNA levels, respectively, were downregulated when mAb1-expressing CHO cells are grown at pH 7.07. FIG. 1C depicts western blot analysis of two mAb1-expressing host cell lines, CHO DG44 and CHO-K1, treated with chemical UPR inducers: tunicamycin and DTT. FIG. 1D depicts qPCR analysis of PDGFRa mRNA levels in both host cell lines of FIG. 1C treated with tunicamycin and DTT. FIG. 1E depicts western blot analysis of mAb1-expressing CHO-K1 cells treated with tunicamycin to activate the UPR in the presence of UPR pathway-specific inhibitors. RT-PCR panel for XBP-1 shows IRE1alpha RNase activation. FIG. 1F depicts qPCR analysis of PDGFRa mRNA levels in CHO-K1 cells treated with tunicamycin in the presence of UPR pathway-specific inhibitors. FIG. 1G depicts western blot analysis of WT and PERK KO empty host CHO-K1 (Clone 9) cell lines treated with tunicamycin and PERK inhibitor.

FIGS. 2A-2E. FIG. 2A depicts western blot analysis of mAb1-expressing CHO-K1 cells treated with thapsigargin to activate the UPR in the presence of different UPR pathway-specific inhibitors. RT-PCR panel of XBP-1 shows IRE1alpha RNase activation. FIG. 2B depicts western blot analysis of empty host CHO-K1 cells treated with Tunicamycin to activate the UPR in the presence of different UPR pathway-specific inhibitors. FIG. 2C depicts qPCR analysis of PDGFRa mRNA levels in CHO-K1 cells treated with thapsigargin in the presence of different UPR pathway-specific inhibitors. FIG. 2D depicts western blot analysis of Cas9-sgRNAs against the PERK gene with a sgRNA against luciferase as control. FIG. 2E depicts western blot analysis of empty host CHO-K1 single cell clones after using Cas9 to knockout PERK. Clone 9 was used in FIG. 1G.

FIGS. 3A-3D. PDGFRa signaling is important for cell growth, e.g., CHO cell growth, and growth factor signaling is intact after PDGFRa inhibition. FIG. 3A is a schematic of PDGFRa and insulin receptor (IR) signaling upstream of protein synthesis, cell cycle progression and cell proliferation. Bolder arrows indicate stronger activation by respective receptors. FIG. 3B depicts empty CHO-K1 host cells VCC and % viability after 4 days in the presence or absence of PDGFRa inhibitor and/or insulin in the seed train media. FIG. 3C depicts western blot analysis of empty host CHO-K1 cells (of FIG. 3B) after 4 days in the presence or absence of PDGFRa inhibitor and/or insulin in the seed train media. FIG. 3D depicts Day 12 relative IVCC, % viability, relative titer and relative Qp of mAb2-expressing CHO-K 1 cells in the presence of PDGFRa inhibitor and/or insulin during production.

FIGS. 4A-4D. FIG. 4A depicts empty host CHO-K1 cells viable cell count (VCC) and % viability after 4 days in increasing PDGFRa inhibitor concentrations in the seed train media. FIG. 4B depicts western blot analysis of empty host CHO-K1 cells after 4 days in increasing PDGFRa inhibitor concentrations in the seed train media. FIG. 4C depicts western blot analysis mAb2-expressing CHO-K 1 cells in production in the presence or absence of PERK inhibitor at 10 μM concentration. FIG. 4D depicts qPCR analysis of downstream targets of PERK branch of UPR, CHOP and GADD34, during production for mAb2-expressing CHO-K 1 cells in the presence or absence of PERK inhibitor.

FIGS. 5A-5C. PDGFRa levels are stabilized during production in PERK KO cell lines. FIG. 5A depicts western blot analysis of mAb2-expressing CHO-K1 single cell clones after using CRISPR-Cas9 to knockout PERK. FIG. 5B depicts Day 14 relative IVCC, % viability, relative titer and relative Qp of mAb2-expressing CHO-K1 PERK KO cells. FIG. 5C depicts western blot analysis of production for mAb2-expressing CHO-K1 WT and PERK KO cells.

FIGS. 6A-6E. PERK and Bax/Bak TKOs synergistically increase bioprocess outcomes. FIG. 6A depicts western blot analysis of mAb3-expressing CHO-K 1 single cell clones in seed train after using Cas9 to knockout PERK. Overall titer, depicted in FIG. 6B, and relative Qp, depicted in FIG. 6C, of various mAb3-expressing CHO-K1 hosts across different bioprocesses: lean production media, rich production media and intensified process using rich production media. FIG. 6D depicts western blot analysis of various mAb3-expressing CHO-K 1 hosts in rich production media. FIG. 6E depicts qPCR analysis of heavy chain and light chain mRNA levels in lean production media and rich production media.

FIGS. 7A-7B. FIG. 7A depicts bioprocess outcomes for a 6-day production of mAb3-expressing pools in either a Bax/Bak DKO background or a PERK/Bax/Bak TKO background showing relative titer, Qp and IVCC. FIG. 7B depicts bioprocess outcomes for a 14-day production of Fab 1-expressing pools in either a WT, PERK KO, Bax/Bak DKO or PERK/Bax/Bak TKO background showing relative titer, Qp and IVCC.

### 5. DETAILED DESCRIPTION

The present disclosure relates to cells (e.g., Chinese Hamster Ovary (CHO) cells) that are modified to reduce or eliminate the expression of certain endogenous proteins, and methods of using such modified cells in the production of a recombinant product of interest, e.g., a recombinant protein, a recombinant viral particle, or a recombinant viral vector. These modifications generate engineered cells with desired traits in several key areas, including improved viability and higher product titers.

One of the commonly used methods for expression of heterologous molecules, such as monoclonal antibodies, is through a fed-batch process, where the cells are initially seeded and then fed in batches over the course of the production period. Different factors during the production process can lead to poor production and product quality outcomes. One factor is the increased proteostatic stress due to the copious synthesis of the heterologous molecule. The endoplasmic reticulum (ER) is able to adapt to this stress by activating the unfolded protein response (UPR). The UPR is typically triggered when the ER reaches its maximal protein-folding capacity and is unable to accommodate the increased demand for protein synthesis and folding. The UPR has three identified ER transmembrane proteins that sense the accumulation of unfolded polypeptides and respond by activating signaling pathways that promote ER homeostasis by expanding the ER, increasing chaperone production and decreasing overall protein translation. When these processes are not able to alleviate the proteostatic stress, sustained UPR activation can lead to apoptotic cell death. During normal conditions, these UPR sensors are bound by an ER chaperone called immunoglobulin binding protein (BiP) in the ER lumen, which keep them inactive. Upon accumulation of unfolded proteins in the ER lumen, BiP dissociates from these sensors to assist with folding of the unfolded or misfolded proteins in order to reduce ER proteostatic stress, thereby allowing activation of UPR receptors.

The three UPR sensors are inositol-requiring enzyme 1 (IRE1), protein kinase R-like ER kinase (PERK), and activating transcription factor 6 (ATF6). The IRE1 branch of the UPR is the most conserved and IRE1 functions both as a kinase and a ribonuclease (RNase). IRE1 is involved in the nonconventional splicing of the XBP1 (X-box binding protein 1) mRNA transcript. The splicing produces the short form of XBP1 and this short form is a transcription factor that regulates UPR target genes to increase the protein folding capacity of ER. These target genes expand and modify the ER through regulating lipid biosynthetic enzymes and ERAD (ER-associated degradation) components. Another branch of the UPR, PERK (Protein Kinase R-like ER Kinase), is a kinase but lacks ribonuclease activity; upon activation PERK reduces overall mRNA translation by phosphorylating elF2alpha, thereby decreasing its activity. At the same time, a subset of mRNAs that contain short open reading frames undergo selective protein translation. One of these mRNAs encodes the activation transcription factor 4 (ATF4), which regulates the expression of another transcription factor called C/EBP homologous protein (CHOP), which regulates components of the apoptotic pathway including Bim and death receptor 5 (DR5). A modest activation of PERK functions cytoprotectively through CHOP, but prolonged PERK activation will lead to cell death. Lastly, the ATF6 branch of the UPR involves the transportation and proteolytic activation of ATF6 from the ER to the Golgi apparatus. Upon proteolysis, ATF6 functions as a transcription factor that helps increase the ER capacity by elevating the production of ER proteins involved in protein folding chaperones such as BiP and GRP94, and foldases such as protein disulfide isomerases. The three branches of the UPR work together to alleviate proteostatic stress by decreasing the overall protein folding load while at the same time increasing the protein folding capacity of the ER.

The present disclosure is based, at least in part, on the discovery that knocking out PERK in a Bax/Bak double knock-out (DKO) antibody-expressing cell line revealed an increase in viability and growth, and surprisingly also showed a synergistic increase in specific productivity and titer as compared to control cell lines.

For clarity, but not by way of limitation, the detailed description of the presently disclosed subject matter is divided into the following subsections:
5.1 Definitions;
5.2 Reduced or Eliminated Expression of Endogenous Proteins;
5.3 Cells Comprising Gene-Specific Modifications;
5.4 Cell Culture Methods;
5.5 Production of a Recombinant Product of Interest; and
5.6 Exemplary Non-Limiting Embodiments

### 5.1 Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “cell culture medium” and “culture medium” refer to a nutrient solution used for growing mammalian cells that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The nutrient solution can optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

“Culturing” a cell refers to contacting a cell with a cell culture medium under conditions suitable to the survival and/or growth and/or proliferation of the cell.

“Batch culture” refers to a culture in which all components for cell culturing (including the cells and all culture nutrients) are supplied to the culturing bioreactor at the start of the culturing process.

“Fed-batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

“Perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.

As used herein, the term “cell,” refers to animal cells, mammalian cells, cultured cells, host cells, recombinant cells and recombinant host cells. Such cells are generally cell lines obtained or derived from animal, e.g., mammalian, tissues which are able to grow and survive when placed in media containing appropriate nutrients and/or growth factors.

The terms “host cell,” “host cell line” and “host cell culture” are used interchangeably and refer to cells and their progeny into which exogenous nucleic acid can be subsequently introduced to create recombinant cells. These host cells may also have been modified (i.e., engineered) to alter or delete the expression of certain endogenous host cell proteins. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line.” The terms “host cell,” “host cell line” and “host cell culture” may also refer to such recombinant cells and their progeny.

The terms “recombinant cell,” “recombinant cell line” and “recombinant cell culture” are used interchangeably and refer to cells and their progeny into which exogenous nucleic acid has been introduced to enable the expression of recombinant product of interest. The recombinant product expressed by such cells may be a recombinant protein, a recombinant viral particle, or a recombinant viral vector.

The term “animal host cell” or “animal cell” refers to cell lines derived from animals that are capable of growth and survival when placed in either monolayer culture or in suspension culture in a medium containing the appropriate nutrients and growth factors. In addition to the mammalian cells described below, examples of suitable animal host cells within the context of the present disclosure can include, but are not limited to, invertebrate and non-mammalian vertebrate (e.g., avian, reptile and amphibian) cells. Examples of invertebrate cells include the following insect cells: Spodoptera frugiperda (caterpillar), Aedes aegypti (mosquito), Aedes albopictus (mosquito), Drosophila melanogaster (fruitfly), and Bombyx mori. See, e.g., Luckow et al., Bio/Technology, 6:47-55 (1988); Miller et al., in Genetic Engineering, Setlow, J. K. et al., eds., Vol. 8 (Plenum Publishing, 1986). pp. 277279; and Maeda et al., Nature, 315:592-594 (1985)

The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells within the context of the present disclosure can include Chinese hamster ovary cells/−DHFR (CHO, Urlaub and Chasm, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). In certain embodiments, the mammalian cells include Chinese hamster ovary cells/-DHFR (CHO, Urlaub and Chasm, Proc. Natl. Acad. Sci. USA, 77:4216 1980); dp12.CHO cells (EP 307,247 published 15 Mar. 1989).

“Growth phase” of the cell culture refers to the period of exponential cell growth (the log phase) where cells are generally rapidly dividing. The duration of time for which the cells are maintained at growth phase can vary based on the cell-type, the rate of growth of cells and/or the culture conditions, for example. In certain embodiments, during this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. In certain embodiments, during the growth phase, cells are cultured in nutrient medium containing the necessary additives generally at about 30°-40° C. in a humidified, controlled atmosphere, such that optimal growth is achieved for the particular cell line. In certain embodiments, cells are maintained in the growth phase for a period of about between one and four days, usually between two to three days.

“Production phase” of the cell culture refers to the period of time during which cell growth is/has plateaued. The logarithmic cell growth typically decreases before or during this phase and protein production takes over. During the production phase, logarithmic cell growth has ended, and protein production is primary. During this period of time the medium is generally supplemented to support continued protein production and to achieve the desired glycoprotein product. Fed-batch and/or perfusion cell culture processes supplement the cell culture medium or provide fresh medium during this phase to achieve and/or maintain desired cell density, viability and/or recombinant protein product titer. A production phase can be conducted at large scale.

The term “activity” as used herein with respect to activity of a protein refers to any activity of a protein including, but not limited to, enzymatic activity, ligand binding, drug transport, ion transport, protein localization, receptor binding, and/or structural activity. Such activity can be modulated, e.g., reduced or eliminated, by reducing or eliminating the expression of the protein, thereby reducing or eliminating the presence of the protein. Such activity can also be modulated, e.g., reduced or eliminated, by altering the nucleic acid sequence encoding the protein such that the resulting modified protein exhibits reduced or eliminated activity relative to a wild type protein.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab,′ Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG and IgM, and several of these can be further divided into subclasses (isotypes), e.g., IgG1, IgG2, IgG3, IgG4, IgA1, and IgA2. In certain embodiments, the antibody is of the IgG1 isotype. In certain embodiments, the antibody is of the IgG2 isotype. The heavy chain constant domains that correspond to the different classes of immunoglobulins are called a, 5, c, y and μ, respectively. The light chain of an antibody can be assigned to one of two types, called kappa (K) and lambda (X), based on the amino acid sequence of its constant domain.

The term “titer” as used herein refers to the total amount of recombinantly expressed antibody produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al, Nature Medicine 2017, published online 12 Jun. 2017, doi:10.1038/nm.4356 or EP 2 101 823 B1).

As used herein, the term “vector” refers to a nucleic acid molecule capable of transporting another nucleic acid to which it has been linked.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 9196 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 8996 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VII domains, respectively. See, e.g., Portolano et al., J. Immunol. 150:880-887 (1993); Clarkson et al., Nature 352:624-628 (1991).

As used herein, the term “cell density” refers to the number of cells in a given volume of medium. In certain embodiments, a high cell density is desirable in that it can lead to higher protein productivity. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor).

As used herein, the term “recombinant protein” refers generally to peptides and proteins, including antibodies, that are encoded by a nucleic acid that is “heterologous,” i.e., foreign to the host cell being utilized, such as a nucleic acid encoding a human antibody that is introduced into a non-human host cell.

As used herein, the term “recombinant viral particle” refers generally to virus particles that may occur naturally or be produced by recombining exogenous nucleic acid for use in vaccine production.

As used herein, the term “recombinant viral vector” refers generally to viral vectors that have been modified to express exogenous viral elements, e.g., for use in gene therapy, including but not limited to recombinant vectors based on adeno-associated virus (AAV), herpes simplex virus (HSV), retrovirus, poxvirus, lentivirus.

### 5.2 Reduced or Eliminated Expression of Endogenous Proteins

In certain embodiments, the present disclosure relates to modified cells, e.g., CHO cells, where the expression of one or more endogenous proteins, is reduced or eliminated. For example, but not by way of limitation, methods for reducing or eliminating endogenous protein expression in a modified cell include: (1) modification of a gene coding for the endogenous protein or component thereof, e.g., by introducing a deletion, insertion, substitution, or combination thereof into the gene; (2) reducing or eliminating the transcription and/or stability of the mRNA encoding the endogenous protein or a component thereof, and (3) reducing or eliminating the translation of the mRNA encoding the endogenous protein or a component thereof. In certain embodiments, the reduction or elimination of protein expression is obtained by targeted genome editing. For example, CRISPR/Cas9-based genome editing can be employed to modify one or more target genes, resulting in the reduction or elimination of expression of the gene (or genes) targeted for editing.

In certain embodiments, one or more of the endogenous cellular proteins targeted for reduced or eliminated expression are selected based on their role in promoting apoptosis. As apoptosis can decrease culture viability and productivity, reducing or eliminating expression of such proteins can positively impact culture viability and productivity. For example, but not by way of limitation, the cellular protein selected based on its role in promoting apoptosis is BCL2 Associated X, Apoptosis Regulator (BAX) or BCL2 Antagonist/Killer 1 (BAK).

In certain embodiments, the modified cells of the present disclosure exhibit reduced or eliminated expression of BAX. In certain embodiments, BAX, as used herein, refers to a eukaryotic BAX cellular protein, e.g., the CHO BAX cellular protein (Entrez Gene ID: 100689032; GenBank ID: EF104643 0.1), and functional variants thereof. In certain embodiments, functional variants of BAX, as used herein encompass BAX sequence variants having 50%, 55%, 60%, 65%, 70%, 75%, 80%, 85%, 90%, 91%, 92%, 93%, 94%, 95%, 96%, 97%, 98%, 99% identity to the wild type BAX sequence of the modified cell used for the production of a recombinant product of interest.

In certain embodiments, the modified cells of the present disclosure exhibit reduced or eliminated expression of BAK. In certain embodiments, BAK, as used herein, refers to a eukaryotic BAK cellular protein, e.g., the CHO BAK cellular protein (GenBank ID: EF104644.1), and functional variants thereof. In certain embodiments, functional variants of BAK, as used herein encompass BAK sequence variants having 50%, 55%, 60%, 65%, 70%, 75%, 80%, 85%, 90%, 91%, 92%, 93%, 94%, 95%, 96%, 97%, 98%, 99% identity to the wild type BAK sequence of the modified cell used for the production of a recombinant product of interest.

In certain embodiments, the modified cells of the present disclosure exhibit reduced or eliminated expression of BAX and BAK.

In certain embodiments, one or more of the endogenous cellular proteins targeted for reduced or eliminated expression are selected based on their role in regulating the unfolded protein response (UPR). For example, but not by way of limitation, the cellular protein selected based on its role in regulating the UPR is inositol-requiring enzyme 1 (IRE1), protein kinase R-like ER kinase (PERK) or activating transcription factor 6 (ATF6). In certain embodiments, the modified cells of the present disclosure exhibit reduced or eliminated expression of PERK. In certain embodiments, PERK, as used herein, refers to a eukaryotic PERK cellular protein, e.g., the CHO PERK cellular protein (Gene ID: 100765343; GenBank: EGW03658.1; and isoforms NCBI Reference Sequence: XP 027285344.2 and NCBI Reference Sequence: XP 016831844.1), and functional variants thereof. In certain embodiments, functional variants of PERK, as used herein encompass PERK sequence variants having 50%, 55%, 60%, 65%, 70%, 75%, 80%, 85%, 90%, 91%, 92%, 93%, 94%, 95%, 96%, 97%, 98%, 99% identity to the wild type PERK sequence of the modified cell used for the production of a recombinant product of interest.

In certain embodiments, the modified cells of the present disclosure exhibit reduced or eliminated expression of the following endogenous proteins: BAX; BAK; and PERK.

In certain embodiments, a cell of the present disclosure is modified to reduce or eliminate the expression of one or more endogenous cellular proteins relative to the expression of the endogenous cellular proteins in an unmodified, i.e., “reference,” cell. In certain embodiments, the reference cells are cells where the expression of one or more particular endogenous proteins, e.g., a BAX; BAK; and/or PERK polypeptide, is not reduced or eliminated. In certain embodiments, a reference cell is a cell that comprises at least one or both wild-type alleles of the gene(s) coding for BAX; BAK; and/or PERK. For example, but not by way of limitation, a reference cell is a cell that has both wild-type alleles of the gene(s) coding for BAX; BAK; and/or PERK. In certain embodiments, the reference cells are WT cells. In certain embodiments, the modification of reducing or eliminating the expression of one or more endogenous cellular proteins is performed before the introduction of the exogenous nucleic acid encoding the recombinant product of interest. In certain embodiments, the modification of reducing or eliminating the expression of one or more endogenous cellular proteins is performed after the introduction of the exogenous nucleic acid encoding the recombinant product of interest.

In certain embodiments, the expression of one or more endogenous proteins, e.g., a BAX; BAK; and/or PERK polypeptide, in a cell that has been modified to reduce or eliminate expression of the endogenous proteins, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding endogenous protein expression of a reference cell, e.g., a WT cell. In certain embodiments, the expression of one or more endogenous proteins in a cell that has been modified to reduce or eliminate expression of the endogenous proteins, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the corresponding endogenous protein expression of a reference cell, e.g., a WT cell.

In certain embodiments, the expression of one or more endogenous proteins, e.g., a BAX; BAK; and/or PERK polypeptide, in a cell that has been modified to reduce or eliminate expression of the endogenous proteins, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2% or at least about 1% of the corresponding endogenous protein expression of a reference cell, e.g., a WT cell. In certain embodiments, the expression of one or more endogenous proteins in a cell that has been modified to reduce or eliminate expression of the endogenous protein, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2%, or at least about 1% of the corresponding endogenous proteins expression of a reference cell, e.g., a WT cell.

In certain embodiments, the expression of one or more particular endogenous proteins, e.g., a BAX; BAK; and/or PERK polypeptide, in a cell that has been modified to reduce or eliminate expression of the endogenous proteins, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous protein expression of a reference cell, e.g., a WT cell. In certain embodiments, the expression of one or more endogenous proteins, e.g., a BAX; BAK; and/or PERK polypeptide, in a cell that has been modified to reduce or eliminate expression of the endogenous proteins, is no more than about 40% of the corresponding endogenous protein expression of a reference cell, e.g., a WT cell. In certain embodiments, the expression of one or more endogenous proteins in a cell that has been modified to reduce or eliminate expression of the endogenous proteins, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous protein expression of a reference cell, e.g., a WT cell.

In certain embodiments, the expression of one or more endogenous proteins, e.g., a BAX; BAK; and/or PERK polypeptide, in a cell that has been modified to reduce or eliminate expression of the endogenous proteins, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous proteins expression of a reference cell, e.g., a WT cell. In certain embodiments, the expression of one or more endogenous proteins, e.g., a BAX; BAK; and/or PERK polypeptide, in a cell that has been modified to reduce or eliminate expression of the endogenous proteins, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous protein expression of a reference cell, e.g., a WT cell.

In certain embodiments, the expression of one or more endogenous proteins, e.g., a BAX; BAK; and/or PERK polypeptide, in a cell that has been modified to reduce or eliminate expression of the endogenous proteins, is between about 5% and about 40% of the corresponding endogenous protein expression of a reference cell, e.g., a WT cell.

In certain embodiments, the expression level of the one or more endogenous proteins, e.g., a BAX; BAK; and/or PERK polypeptide, in different reference cells (e.g., cells that comprise at least one or both wild-type alleles of the corresponding gene) can vary.

In certain embodiments, a genetic engineering system is employed to reduce or eliminate the expression of one or more particular endogenous protein (e.g., a BAX; BAK; and/or PERK expression). Various genetic engineering systems known in the art can be used for the methods disclosed herein. Non-limiting examples of such systems include the CRISPR/Cas system, the zinc-finger nuclease (ZFN) system, the transcription activator-like effector nuclease (TALEN) system and the use of other tools for reducing or eliminating protein expression by gene silencing, such as small interfering RNAs (siRNAs), short hairpin RNA (shRNA), and microRNA (miRNA). Any CRISPR/Cas systems known in the art, including traditional, enhanced or modified Cas systems, as well as other bacterial based genome excising tools such as Cpf-1 can be used with the methods disclosed herein.

In certain embodiments, a portion of one or more genes, e.g., genes coding for an endogenous protein such as BAX; BAK; and/or PERK polypeptides, is deleted to reduce or eliminate expression of the corresponding endogenous protein in a cell. In certain embodiments, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the gene is deleted. In certain embodiments, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the gene is deleted. In certain embodiments, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the gene is deleted.

In certain embodiments, at least one exon of a gene encoding a BAX; BAK; and/or PERK polypeptide is at least partially deleted in a cell. “Partially deleted,” as used herein, refers to at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, no more than about 95%, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of a region, e.g., of the exon, is deleted.

In certain non-limiting embodiments, a CRISPR/Cas9 system is employed to reduce or eliminate the expression of one or more endogenous proteins, e.g., a BAX; BAK; and/or PERK polypeptide in a cell. A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

CRISPR/Cas9 strategies can employ a vector to transfect a cell. The guide RNA (gRNA) can be designed for each application as this is the sequence that Cas9 uses to identify and directly bind to the target DNA in a cell. Multiple crRNAs and the tracrRNA can be packaged together to form a single-guide RNA (sgRNA). The sgRNA can be joined together with the Cas9 gene and made into a vector in order to be transfected into cells.

In certain embodiments, the CRISPR/Cas9 system for use in reducing or eliminating the expression of one or more endogenous proteins, e.g., a BAX; BAK; and/or PERK polypeptide, comprises a Cas9 molecule and one or more gRNAs comprising a targeting domain that is complementary to a target sequence of the gene encoding the endogenous protein or a component thereof. In certain embodiments, the target gene is a region of the gene coding for the endogenous protein, e.g., a BAX; BAK; and/or PERK polypeptide. The target sequence can be any exon or intron region within the gene.

In certain embodiments, the gRNAs are administered to the cell in a single vector and the Cas9 molecule is administered to the cell in a second vector. In certain embodiments, the gRNAs and the Cas9 molecule are administered to the cell in a single vector. Alternatively, each of the gRNAs and Cas9 molecule can be administered by separate vectors. In certain embodiments, the CRISPR/Cas9 system can be delivered to the cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). In certain embodiments, administering the CRISPR/Cas9 system to the cell results in the reduction or elimination of the expression of an endogenous protein, e.g., a BAX; BAK; and/or PERK polypeptide.

In certain embodiments, the genetic engineering system is a ZFN system for reducing or eliminating the expression of one or more particular endogenous protein in a cell, e.g., a BAX; BAK; and/or PERK polypeptide. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type its restriction endonuclease Fokl. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)).

In certain embodiments, the genetic engineering system is a TALEN system for reducing or eliminating the expression of one or more particular endogenous protein, e.g., a BAX; BAK; and/or PERK polypeptide, in a cell. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)). In certain embodiments, the target gene encodes a BAX; BAK; and/or PERK.

In certain embodiments, the expression of one or more particular endogenous protein, e.g., a BAX; BAK; and/or PERK polypeptide, can be reduced or eliminated using oligonucleotides that have complementary sequences to corresponding nucleic acids (e.g., mRNA). Non-limiting examples of such oligonucleotides include small interference RNA (siRNA), short hairpin RNA (shRNA), and micro RNA (miRNA). In certain embodiments, such oligonucleotides can be homologous to at least a portion of a BAX; BAK; and/or PERK nucleic acid sequence, wherein the homology of the portion relative to the corresponding nucleic acid sequence is at least about 75 or at least about 80 or at least about 85 or at least about 90 or at least about 95 or at least about 98 percent. In certain non-limiting embodiments, the complementary portion can constitute at least 10 nucleotides or at least 15 nucleotides or at least 20 nucleotides or at least 25 nucleotides or at least 30 nucleotides and the antisense nucleic acid, shRNA, mRNA or siRNA molecules can be up to 15 or up to 20 or up to 25 or up to 30 or up to 35 or up to 40 or up to 45 or up to 50 or up to 75 or up to 100 nucleotides in length. Antisense nucleic acid, shRNA, mRNA or siRNA molecules can comprise DNA or atypical or non-naturally occurring residues, for example, but not limited to, phosphorothioate residues.

The genetic engineering systems disclosed herein can be delivered into the cell using a viral vector, e.g., retroviral vectors such as gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the cells disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic engineering of the cells disclosed herein. For example, a nucleic acid molecule can be introduced into the cells by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

### 5.3 Cells Comprising Gene-Specific Modifications

In one aspect, the present disclosure relates to cells or compositions comprising one or more cells, e.g., animal cells, having reduced or eliminated expression of one or more endogenous proteins. In certain embodiments, the cell has reduced or eliminated expression of a BAX; BAK; and/or PERK polypeptide.

As used herein, eliminated expression refers to the elimination of the expression of a particular endogenous protein, e.g., a BAX; BAK; and/or PERK polypeptide, in the cell as compared to a reference cell. As used herein, reduced expression refers to a reduction in the expression of an endogenous protein, e.g., a BAX; BAK; and/or PERK polypeptide, in the cell as compared to a reference cell.

Non-limiting examples of cells useful in connection with the subject matter of the present disclosure include invertebrate and non-mammalian vertebrate (e.g., avian, reptile and amphibian) cells, e.g., Spodoptera frugiperda cells, Aedes aegypti cells, Aedes albopictus cells, Drosophila melanogaster cells, and Bombyx mori cells, or mammalian cells, e.g., CHO cells (e.g., DHFR CHO cells), dp12.CHO cells, CHO-K1 (ATCC, CCL-61), monkey kidney CV1 line transformed by SV40 (e.g., COS-7 ATCC CRL-1651), human embryonic kidney line (e.g., HEK 293 or HEK 293 cells subcloned for growth in suspension culture), baby hamster kidney cells (e.g., BHK, ATCC CCL 10), mouse sertoli cells (e.g. TM4), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, BB 8065), mouse mammary tumor (e.g., MMT 060562, ATCC CCL51), TRI cells, MRC 5 cells, FS4 cells, human hepatoma line (e.g., Hep G2), myeloma cell lines (e.g., YO, NS0 and Sp2/0). In certain embodiments, the cells are CHO cells. Additional non-limiting examples of CHO cells include CHO K1SV cells, CHO DG44 cells, a CHO DUKXB-11 cells, CHOK1S cells and CHO KIM cells.

In certain embodiments, the cells disclosed herein express a recombinant product of interest. In certain embodiments, the recombinant product of interest is a recombinant protein. In certain embodiments, the recombinant product of interest is a monoclonal antibody. Additional non-limiting examples of recombinant products of interest are provided in Section 5.5.

In certain embodiments, the cells disclosed herein can be used for production of commercially useful amounts of the recombinant product of interest. In certain embodiments, the cells disclosed herein facilitate the production of commercially useful amounts of a recombinant product of interest, at least in part, via higher productivity and higher titers and increased/extended viability, relative to a reference cells, e.g., WT cells.

In certain embodiments, the cells disclosed herein can comprise a nucleic acid that encodes a recombinant product of interest. In certain embodiments, the nucleic acid can be present in one or more vectors, e.g., expression vectors. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a cell into which they are introduced (e.g., bacterial vectors having a bacterial origin of replication and episomal mammalian vectors). Other vectors (e.g., non-episomal mammalian vectors) are integrated into the genome of a cell upon introduction into the cell, and thereby are replicated along with the cellular genome. Moreover, certain vectors, expression vectors, are capable of directing the expression of genes to which they are operably linked. In general, expression vectors of utility in recombinant DNA techniques are often in the form of plasmids (vectors). Additional non-limiting examples of expression vectors for use in the present disclosure include viral vectors (e.g., replication defective retroviruses, adenoviruses and adeno-associated viruses) that serve equivalent functions.

In certain embodiments, the nucleic acid encoding a recombinant product of interest can be introduced into a cell, disclosed herein. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, the cell is eukaryotic, e.g., a Chinese Hamster Ovary (CHO) cell. In certain embodiments, the cell is a lymphoid cell (e.g., YO, NSO, Sp20 cell).

In certain embodiments, the nucleic acid encoding a recombinant product of interest can be randomly integrated into a host cell genome (“Random Integration” or “RI”). For example, but not by way of limitation, a nucleic acid encoding a recombinant product of interest can be randomly integrated into the genome of a cell that has also been modified to have reduced or eliminated expression of one or more particular endogenous proteins, e.g., a BAX; BAK; and/or PERK polypeptide.

In certain embodiments, the nucleic acid encoding a recombinant product of interest can be integrated into a host cell genome in a targeted manner (“Targeted Integration” or “TP”, as described in detail herein). For example, but not by way of limitation, a nucleic acid encoding a recombinant product of interest can be integrated in a targeted manner into the genome of a cell that has been modified to have reduced or eliminated expression of one or more particular endogenous protein, e.g., a BAX; BAK; and/or PERK polypeptide. In certain embodiments, the use of a TI host cell for the introduction of a nucleic acid encoding a recombinant product of interest will provide for robust, stable cell culture performance and lower risk of sequence variants in the resulting recombinant product of interest. TI host cells and strategies for the use of the same are described in detail in U.S. Patent Application Publication No. US20210002669, the contents of which are incorporated by reference in their entirety.

In certain embodiments employing targeted integration, the exogenous nucleotide sequence is integrated at a site within a specific locus of the genome of a TI host cell. In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleotide sequence immediately 5′ of the integrated exogenous sequence is selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW 003616412.1, nucleotides 293481-315265 of NW_003615063.1, nucleotides 2650443-2662054 of NW_006882936.1, or nucleotides 82214-97705 of NW_003615411.1 and sequences at least 50% homologous thereto. In certain embodiments, the nucleotide sequence immediately 5′ of the integrated exogenous sequence are at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW 006882936.1, or nucleotides 82214-97705 of NW 003615411.1.

In certain embodiments, the nucleotide sequence immediately 3′ of the integrated exogenous sequence is selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-3 62442 of NW_003615063.1, nucleotides 2662055-2701768 of NW 006882936.1, or nucleotides 97706-105117 of NW_003615411.1 and sequences at least 50% homologous thereto. In certain embodiments, the nucleotide sequence immediately 3′ of the integrated exogenous sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW_006884592.1, nucleotides 491910667813 of NW_006881296.1, nucleotides 79769-100059 of NW_003616412.1, nucleotides 315266-362442 of NW 003615063.1, nucleotides 2662055-2701768 of NW_006882936.1, or nucleotides 97706-105117 of NW 003615411.1.

In certain embodiments, the integrated exogenous sequence is flanked 5′ by a nucleotide sequence selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. and sequences at least 50% homologous thereto. In certain embodiments, the integrated exogenous sequence is flanked 3′ by a nucleotide sequence selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1 and sequences at least 50% homologous thereto. In certain embodiments, the nucleotide sequence flanking 5′ of the integrated exogenous nucleotide sequence is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481315265 of NW_003615063.1, nucleotides 2650443-2662054 of NW_006882936.1, and nucleotides 82214-97705 of NW_003615411.1. In certain embodiments, the nucleotide sequence flanking 3′ of the integrated exogenous nucleotide sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW 006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1.

In certain embodiments, the integrated exogenous nucleotide sequence is operably linked to a nucleotide sequence selected from the group consisting of Contigs NW 006874047.1, NW_006884592.1, NW_006881296.1, NW_003616412.1, NW_003615063.1, NW_006882936.1, and NW_003615411.1 and sequences at least 50% homologous thereto. In certain embodiments, the nucleotide sequence operably linked to the exogenous nucleotide sequence is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleic acid encoding a product of interest can be integrated into a host cell genome using transposase-based integration. Transposase-based integration techniques are disclosed, for example, in Trubitsyna et al., Nucleic Acids Res. 45(10):e89 (2017), Li et al., PNAS 110(25):E2279-E2287 (2013) and WO 2004/009792, which are incorporated by reference herein in their entireties.

In certain embodiments, the nucleic acid encoding a recombinant product of interest can be randomly integrated into a host cell genome (“Random Integration” or “RP”). In certain embodiments, the random integration can be mediated by any method or systems known in the art. In certain embodiments, the random integration is mediated by MaxCyte STX® electroporation system.

In certain embodiments, targeted integration can be combined with random integration. In certain embodiments, the targeted integration can be followed by random integration. In certain embodiments, random integration can be followed by targeted integration. For example, but not by way of limitation, a nucleic acid encoding a recombinant product of interest can be randomly integrated into the genome of a cell that has been modulated to have reduced or eliminated expression of one or more particular endogenous protein, e.g., a BAX; BAK; and/or PERK, and a nucleic acid encoding the same recombinant product of interest can be integrated in the genome of the cell in a targeted manner.

In certain embodiments, the cells disclosed herein comprise one or more altered genes. In certain embodiments, the alteration to the gene reduces or eliminates the expression of an endogenous protein. In certain embodiments, the cells disclosed herein comprise one or more altered BAX; BAK; and/or PERK genes. In certain embodiments, the subsequent transcript of an altered BAX; BAK; and/or PERK gene codes for an endogenous protein having reduced or eliminated expression. In certain embodiments, the one or more altered genes are altered by disruption of a coding region. In certain embodiments, the genes alteration comprises a biallelic alteration. In certain embodiments, the genes alteration comprises a deletion of 1 or more base pairs, 2 or more base pairs, 3 or more base pairs, 4 or more base pairs, 5 or more base pairs, 6 or more base pairs, 7 or more base pairs, 8 or more base pairs, 9 or more base pairs, 10 or more base pairs, 11 or more base pairs, 12 or more base pairs, 13 or more base pairs, 14 or more base pairs, 15 or more base pairs, 16 or more base pairs, 17 or more base pairs, 18 or more base pairs, 19 or more base pairs, or 20 or more base pairs.

In certain embodiments, the present disclosure relates to modified cells or compositions comprising one or more modified cells, where the modified cells or compositions comprising one or more modified cells exhibit one or more of the following features: 1) the modified cell exhibits improved cell culture performance relative to similar cells lacking the modification; and 2) the modified cells exhibit improved viability relative to similar cells lacking the modification.

In certain embodiments, the present disclosure relates to cells or compositions comprising one or more cells having all of the following features: 1) the modified cell exhibits improved cell culture performance relative to similar cells lacking the modification; and 2) the modified cells exhibit improved viability relative to similar cells lacking the modification.

In certain embodiments, the modified cells of the present disclosure exhibit improved cell culture performance relative to similar cells lacking the modification. In certain embodiments, the modified cells of the present disclosure exhibit improved cell culture performance due to: i) increased/extended viability and healthier mitochondria for metabolism; and/or ii) higher productivity and higher titers. In certain embodiments, the modified cells of the present disclosure exhibit increased/extended viability and healthier mitochondria for metabolism, due to reduced or eliminated expression of BAX; BAK and/or PERK. In certain embodiments, the modified cells of the present disclosure exhibit increased/extended viability and healthier mitochondria for metabolism, due to reduced or eliminated expression of BAX. In certain embodiments, the modified cells of the present disclosure exhibit increased/extended viability and healthier mitochondria for metabolism, due to reduced or eliminated expression of BAK. In certain embodiments, the modified cells of the present disclosure exhibit higher productivity and higher titers, due to reduced or eliminated expression of PERK. In certain embodiments, the modified cells of the present disclosure exhibit increased/extended viability and improved cell culture performance due to reduced or eliminated expression of BAX; BAK, and/or PERK.

In certain embodiments, the present disclosure relates to modified cells or compositions comprising one or more TI cells exhibiting improved cell culture performance. In certain embodiments, the TI cells of the present disclosure exhibit reduced or eliminated expression of BAX; BAK; and PERK. In certain embodiments, the TI cells of the present disclosure exhibit reduced or eliminated expression of BAX. In certain embodiments, the TI cells of the present disclosure exhibit reduced or eliminated expression of BAK. In certain embodiments, the TI cells of the present disclosure exhibit reduced or eliminated expression of PERK.

In certain embodiments, a cell is a cell line. In certain embodiments, a cell is a cell line that has been cultured for a certain number of generations. In certain embodiments, a cell is a primary cell.

In certain embodiments, expression of a polypeptide of interest is stable if the expression level is maintained at certain levels, increases, or decreases less than 20%, over 10, 20, 30, 50, 100, 200, or 300 generations. In certain embodiments, expression of a polypeptide of interest is stable if the culture can be maintained without any selection. In certain embodiments, expression of a polypeptide of interest is high if the polypeptide product of the gene of interest reaches about 1 g/L, about 2 g/L, about 3 g/L, about 4 g/L, about 5 g/L, about 10 g/L, about 12 g/L, about 14 g/L, or about 16 g/L.

Exogenous nucleotides of interest or vectors can be introduced into a host cell by conventional cell biology methods including, but not limited to, transfection, transduction, electroporation, or injection. In certain embodiments, exogenous nucleotides of interest or vectors are introduced into a host cell by chemical-based transfection methods comprising lipid-based transfection method, calcium phosphate-based transfection method, cationic polymer-based transfection method, or nanoparticle-based transfection. In certain embodiments, exogenous nucleotides of interest are introduced into a host cell by virus-mediated transduction including, but not limited to, lentivirus, retrovirus, adenovirus, or adeno-associated virus-mediated transduction. In certain embodiments, exogenous nucleotides of interest or vectors are introduced into a host cell via gene gun-mediated injection. In certain embodiments, both DNA and RNA molecules are introduced into a host cell using methods described herein.

### 5.4 Cell Culturing Methods

In one aspect, the present disclosure provides a method for producing a recombinant product of interest comprising culturing a modified cell disclosed herein. Suitable culture conditions for mammalian cells known in the art can be used for culturing the modified cells disclosed herein (J. Immunol. Methods (1983) 56:221-234) or can be easily determined by the skilled artisan (see, for example, Animal Cell Culture: A Practical Approach 2nd Ed., Rickwood, D. and Hames, B. D., eds. Oxford University Press, New York (1992)).

Cell culture can be prepared in a medium suitable for the particular cell being cultured. Commercially available media such as Ham's F10 (Sigma), Minimal Essential Medium (MEM, Sigma), RPMI-1640 (Sigma) and Dulbecco's Modified Eagle's Medium (DMEM, Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or U.S. Pat. No. 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin (gentamicin), trace elements (defined as inorganic compounds usually present at final concentrations in the micromolar range) lipids (such as linoleic or other fatty acids) and their suitable carriers, and glucose or an equivalent energy source. Any other necessary supplements can also be included at appropriate concentrations that would be known to those skilled in the art.

In certain embodiments, the cell that has been modified to reduce and/or eliminate the activity of a particular endogenous protein is a CHO cell. Any suitable medium can be used to culture the CHO cell of the present disclosure. In certain embodiments, a suitable medium for culturing the CHO cell can contain a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone HS or Primatone RL (Sheffield, England), or the equivalent; a cell protective agent, such as Pluronic F68 or the equivalent pluronic polyol; gentamycin; and trace elements.

In certain embodiments, the cell that has been modified to reduce and/or eliminate the expression of a particular endogenous protein, e.g. a BAX; BAK; and/or PERK polypeptide, is a cell that expresses a recombinant product. The recombinant product can be produced by growing cells which express the recombinant product of interest under a variety of cell culture conditions. For instance, cell culture procedures for the large or small-scale production of recombinant products are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode.

In certain embodiments, the cell culture of the present disclosure is performed in a stirred tank bioreactor system and a fed batch culture procedure is employed. In the fed batch culture, the cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

In certain embodiments, the cells of the culture can be propagated according to any scheme or routine that can be suitable for the specific cell and the specific production plan contemplated. Therefore, the present disclosure contemplates a single step or multiple step culture procedure. In a single step culture, the cells are inoculated into a culture environment and the processes of the instant disclosure are employed during a single production phase of the cell culture. Alternatively, a multi-stage culture is envisioned. In the multi-stage culture cells can be cultivated in a number of steps or phases. For instance, cells can be grown in a first step or growth phase culture wherein cells, possibly removed from storage, are inoculated into a medium suitable for promoting growth and high viability. The cells can be maintained in the growth phase for a suitable period of time by the addition of fresh medium to the cell culture.

In certain embodiments, fed batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (d02) and the like, are those used with the particular host and will be apparent to the ordinarily skilled artisan. Generally, the pH is adjusted to a level between about 6.5 and 7.5 using either an acid (e.g., CO2) or a base (e.g., Na2CO3 or NaOH). A suitable temperature range for culturing mammalian cells such as CHO cells is between about 30° to 38° C. and a suitable d02 is between 5-90% of air saturation.

At a particular stage the cells can be used to inoculate a production phase or step of the cell culture. Alternatively, as described above the production phase or step can be continuous with the inoculation or growth phase or step.

In certain embodiments, the culturing methods described in the present disclosure can further include harvesting the recombinant product from the cell culture, e.g., from the production phase of the cell culture. In certain embodiments, the recombinant product produced by the cell culture methods of the present disclosure can be harvested from the third bioreactor, e.g., production bioreactor. For example, but not by way of limitation, the disclosed methods can include harvesting the recombinant product at the completion of the production phase of the cell culture. Alternatively, or additionally, the recombinant product can be harvested prior to the completion of the production phase. In certain embodiments, the recombinant product can be harvested from the cell culture once a particular cell density has been achieved. For example, but not by way of limitation, the cell density can be from about 2.0×10′ cells/mL to about 5.0×10′ cells/mL prior to harvesting.

In certain embodiments, harvesting the product from the cell culture can include one or more of centrifugation, filtration, acoustic wave separation, flocculation and cell removal technologies.

In certain embodiments, the recombinant product of interest can be secreted from the cells or can be a membrane-bound, cytosolic or nuclear protein. In certain embodiments, soluble forms of the recombinant product can be purified from the conditioned cell culture media and membrane-bound forms of the recombinant product can be purified by preparing a total membrane fraction from the expressing cells and extracting the membranes with a nonionic detergent such as TRITON® X-100 (EMD Biosciences, San Diego, Calif.). In certain embodiments, cytosolic or nuclear proteins can be prepared by lysing the cells (e.g., by mechanical force, sonication and/or detergent), removing the cell membrane fraction by centrifugation and retaining the supernatant.

### 5.5 Production of a Recombinant Product of Interest

The cells and/or methods of the present disclosure can be used to produce any recombinant product of interest that can be expressed by the cells disclosed herein.

5.5.1.1 Viral Particle and Viral Vector Products

In certain embodiments, the cells and/or methods of the present disclosure can be used for the production of viral particles or viral vectors. In certain embodiments, the methods of the present disclosure can be used for the production of viral particles. In certain embodiments, the methods of the present disclosure can be used for the production of viral vectors. In certain embodiments, the methods of the present disclosure can be used for the expression of virus polypeptides. Non-limiting examples of such polypeptides include virus proteins, virus structural (Cap) proteins, virus packaging (Rep) proteins, AAV capsid proteins and virus helper proteins. In certain embodiments, the virus polypeptide is an AAV virus polypeptide.

In certain embodiments, the cells useful in connection with the production of viral particles or viral vectors include, but are not limited to: human embryonic kidney line (e.g., HEK 293 or HEK 293 cells subcloned for growth in suspension culture), human cervical carcinoma cells (e.g., HELA, ATCC CCL 2), human lung cells (e.g., W138, ATCC CCL 75), human liver cells (e.g., Hep G2, HB 8065), human hepatoma line (e.g., Hep G2), myeloma cell lines (e.g., YO, NS0 and Sp2/0), monkey kidney CV1 line transformed by SV40 (e.g., COS-7 ATCC CRL-1651), baby hamster kidney cells (e.g., BHK, ATCC CCL 10), mouse sertoli cells (e.g. TM4), monkey kidney cells (e.g., CV1 ATCC CCL 70), African green monkey kidney cells (e.g., VERO-76, ATCC CRL-1587), canine kidney cells (e.g., MDCK, ATCC CCL 34), buffalo rat liver cells (e.g., BRL 3A, ATCC CRL 1442), mouse mammary tumor (e.g., MMT 060562, ATCC CCL51), TM cells, MRC 5 cells, and FS4 cells. In certain embodiments, the cells are CHO cells. Additional non-limiting examples of CHO cells include CHO K1SV cells, CHO DG44 cells, a CHO DUKXB-1 1 cells, CHOKIS cells and CHO KIM cells In certain embodiments, examples of genes of interest that can be carried by the viral particles produced by the methods describe herein include mammalian polypeptides, such as, e.g., renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; leptin; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hematopoietic growth factor; tumor necrosis factor-alpha and -beta; a tumor necrosis factor receptor such as death receptor 5 and CD120; TNF-related apoptosis-inducing ligand (TRAIL); B-cell maturation antigen (BCMA); B-lymphocyte stimulator (BLyS); a proliferation-inducing ligand (APRIL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; platelet-derived endothelial cell growth factor (PD-ECGF); a vascular endothelial growth factor family protein (e.g., VEGF-A, VEGF-B, VEGF-C, VEGF-D, and P1GF); a platelet-derived growth factor (PDGF) family protein (e.g., PDGF-A, PDGF-B, PDGF-C, PDGF-D, and dimers thereof); fibroblast growth factor (FGF) family such as aFGF, bFGF, FGF4, and FGF9; epidermal growth factor (EGF); receptors for hormones or growth factors such as a VEGF receptor(s) (e.g., VEGFR1, VEGFR2, and VEGFR3), epidermal growth factor (EGF) receptor(s) (e.g., ErbB1, ErbB2, ErbB3, and ErbB4 receptor), platelet-derived growth factor (PDGF) receptor(s) (e.g., PDGFR-α and PDGFR-P), and fibroblast growth factor receptor(s); TIE ligands (Angiopoietins, ANGPT1, ANGPT2); Angiopoietin receptor such as TIE1 and TIE2; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); a chemokine such as CXCL12 and CXCR4; an interferon such as interferon-alpha, -beta, and -gamma; colony stimulating factors (CSFs), e.g., M-CSF, GM-CSF, and G-CSF; a cytokine such as interleukins (ILs), e.g., IL-1 to IL-10; midkine; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD 111a, CD 11b, CD 11 c, CD18, an ICAM, VLA-4 and VCAM; ephrins; Bv8; Delta-like ligand 4 (DLL4); Del-1; BMP9; BMP10; Follistatin; Hepatocyte growth factor (HGF)/scatter factor (SF); Alkl; Robo4; ESM1; Perlecan; EGF-like domain, multiple 7 (EGFL7); CTGF and members of its family; thrombospondins such as thrombospondini and thrombospondin2; collagens such as collagen IV and collagen XVIII; neuropilins such as NRP1 and NRP2; Pleiotrophin (PTN); Progranulin; Proliferin; Notch proteins such as Notchi and Notch4; semaphorins such as Sema3A, Sema3C, and Sema3F; a tumor associated antigen such as CA125 (ovarian cancer antigen); immunoadhesins; and fragments and/or variants of any of the above-listed polypeptides as well as antibodies, including antibody fragments, binding to one or more protein, including, for example, any of the above-listed proteins.

In some embodiments, the gene of interest carried by the viral particles produced by the mammalian cells of the present disclosure may encode proteins that bind to, or interact with, any protein, including, without limitation, cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (aFGF), FGF2 ((3FGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FELL (EPSELON), FEL1 (ZETA), IL 1A, IL 1B, Th2, IL3, IL4, IL5, IL6, IL7, IL8, IL9, IL 10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B,11,29, IL30, PDGFA, PDGFB, TGFA, TGFB1, TGFB2, TGFBb3, LTA (TNF-(3), LTB, TNF (TNF-a), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (AP03L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 1IRA, IL12RB1, IL12RB2, 1L13RA1, IL13RA2, IL15RA, IL 17R, IL 18R1, IL20RA, IL21R, IL22R, IL1HY1, IL TRAP, IL1RAPL1, IL1RAPL2, IL1RN, IL6ST, IL18BP, IL18RAP, IL22RA2, AlF1, HGF, LEP (leptin), PTN, and THPO.k.

In some embodiments, the gene of interest carried by the viral particles produced by the mammalian cells of the present disclosure may encode proteins that bind to, or interact with, a chemokine, chemokine receptor, or a chemokine-related protein selected from the group consisting of CCLI (1-309), CCL2 (MCP-1/MCAF), CCL3 (MIP-Iα), CCL4 (MIP-Iα), CCLS (RANTES), CCL7 (MCP-3), CCL8 (mcp-2), CCL11 (eotaxin), CCL13 (MCP-4), CCL15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8R(3), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL. In some embodiments, the polypeptide expressed by the mammalian cells of the present disclosure may bind to, or interact with, 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAIL; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP16); CCL16 (HCC-4); CCL17 (TARC); CCL18 (PARC); CCL19 (MIP-3(3); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Ia); CCL4 (MDP-I (3); CCL5 (RANTES); CCL7 (MCP-3); CCL8 (mcp-2); CCNA1; CCNA2; CCND1; CCNE1; CCNE2; CCR1 (CKRI/HM145); CCR2 (mcp-IR(3/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLF SF3; CKLF SF4; CKLFSF5; CKLF SF6; CKLF SF 7; CKLF SF 8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FELL (EPSILON); FILL (ZETA); FLJ12584; FLJ25530; FLRTI (fibronectin); FLT1; FOS; FOSL1 (FRA-1); FY (DARC); GABRP (GABAa); GAGEBI; GAGECI; GALNAC4S-6ST; GATA3; GDF5; GDNF-Ra1 (GDNF family receptor alpha 1; GFRA1; GDNFR; GDNFRA; RETL1; TRNR1; RETIL; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOPI; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF IR; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; ILIA; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, IL1RN; IL2; IL20; IL20Ra; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); a407 and aEr37 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLKS; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67, RIG-E, SCA-2, TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3 C2; NR4A1; NR4A2; NR4A3; NR5A1; NR5A2; NR6A1; NRP1; NRP2; NT5E; NTN4; ODZI; OPRD1; 0X40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PM; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF 110 (ZNF 144); ROB O2; S100A2; SCGB1D2 (lipophilin B); SCGB2A1 (mammaglobin2); SCGB2A2 (mammaglobin1); SCYEI (endothelial Monocyte-activating cytokine); SDF2; Sema 5b (FLJ10372, KIAA1445, Mm.42015, SEMASB, SEMAG, Semaphorin 5b Hlog, sema domain, seven thrombospondin repeats (type 1 and type 1-like), transmembrane domain (TM) and short cytoplasmic domain, (semaphorin) 5B); SEMINA1; SERPINA3; SERP1NB 5 (maspin); SERPINE1 (PAI-1); SERPDMF 1; SHBG; SLA2; SLC2A2; SLC33A1; SLC43A1; SLIT2; SPPI; SPRR1B (Spr1); ST6GAL1; STABI; STAT6; STEAP (six transmembrane epithelial antigen of prostate); STEAP2 (HGNC 8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSFlA; TNFRSFlB; TNFRSF21; TNFRSFS; TNFRSF6 (Fas); TNFRSF7; TNFRSF8; TNFRSF9; TNF SF 10 (TRAIL); TNF SF11 (TRANCE); TNF SF12 (AP03L); TNF SF13 (April); TNF SF13B; TNF SF 14 (HVEM-L); TNF SF 15 (VEGI); TNFSF18; TNFSF4 (OX40 ligand); TNFSFS (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; F1114627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-1b); XCRI (GPR5/CCXCRI); YY1; and/or ZFPM2.

Many other virus components and/or other genes of interest may be packaged by the mammalian cells in accordance with the present disclosure, and the above lists are not meant to be limiting.

### 5.5.1.2 Recombinant Protein Products

In certain embodiments, the cells and/or methods of the present disclosure can be used for the production of recombinant proteins, e.g., recombinant mammalian proteins. Non-limiting examples of such recombinant proteins include hormones, receptors, fusion proteins including antibody fusion proteins (for e.g., antibody-cytokine fusion proteins), regulatory factors, growth factors, complement system factors, enzymes, clotting factors, anti-clotting factors, kinases, cytokines, CD proteins, interleukins, therapeutic proteins, diagnostic proteins and antibodies. The cells and/or methods of the present disclosure are not specific to the molecule, e.g., antibody, that is being produced.

In certain embodiments, the methods of the present disclosure can be used for the production of antibodies, including therapeutic and diagnostic antibodies or antigen-binding fragments thereof. In certain embodiments, the antibody produced by cell and methods of the present disclosure can be, but are not limited to, monospecific antibodies (e.g., antibodies consisting of a single heavy chain sequence and a single light chain sequence, including multimers of such pairings), multispecific antibodies and antigen-binding fragments thereof For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof

### 5.5.1.3 Multispecific Antibodies

In certain aspects, an antibody produced by cells and methods provided herein is a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). In certain aspects, the multispecific antibody has three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539).

Multispecific antibodies can also be provided in an asymmetric form with a domain crossover in one or more binding arms of the same antigen specificity, i.e., by exchanging the VH/VL domains (see, e.g., WO 2009/080252 and WO 2015/150447), the CH1/CL domains (see, e.g., WO 2009/080253) or the complete Fab arms (see, e.g., WO 2009/080251, WO 2016/016299, also see Schaefer et al, PNAS, 108 (2011) 1187-1191, and Klein at al., MAbs 8 (2016) 1010-20). In certain embodiments, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

In certain embodiments, particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot. Eng. 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 5.5.1.4 Antibody Fragments

In certain aspects, an antibody produced by the cells and methods provided herein is an antibody fragment. For example, but not by way of limitation, the antibody fragment is a Fab, Fab′, Fab′-SH or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

In certain embodiments, the antibody fragment is a diabody, a triabody or a tetrabody. “Diabodies” are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

In certain aspects, an antibody fusion protein produced by the cells and methods provided herein is an antibody-cytokine fusion protein. While such antibody-cytokine fusion proteins can comprise full length antibodies, the antibody of the antibody-cytokine fusion protein is, in certain embodiments, an antibody fragment, e.g., a single-chain variable fragment (scFv), a diabodies, aFab fragment, or a small immunoprotein (SIP). In certain embodiments, the cytokine can be fused to the N-terminus or the C-terminus of the antibody. In certain embodiments, the cytokine of the antibody-cytokine fusion protein consists of multiple subunits. In certain embodiments, the subunits of the cytokine are the same (homomeric). In certain embodiments, the subunits of the cytokine are the distinct (heterometic). In certain embodiments, the subunits of the cytokine are fused to the same antibody. In certain embodiments, the subunits of the cytokine are fused to a different antibody. For a review of antibody-cytokine fusion protein, see, e.g., Murer et al., N Biotechnol., 52: 42-53 (2019).

Antibody fragments can be made by various techniques, including but not limited to proteolytic digestion of an intact antibody.

### 5.5.1.5 Chimeric and Humanized Antibodies

In certain aspects, an antibody produced by the cells and methods provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5.5.1.6 Human Antibodies

In certain aspects, an antibody produced by the cells and methods provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-I125 (2005).

See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### 5.5.1.7 Target Molecules

Non-limiting examples of molecules that can be targeted by an antibody produced by the cells and methods disclosed herein include soluble serum proteins and their receptors and other membrane bound proteins (e.g., adhesins). In certain embodiments, an antibody produced by the cells and methods disclosed herein is capable of binding to one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (aFGF), FGF2 ((3FGF), FGF3 (int-2), FGF4 (HST), FGF5, FGF6 (HST-2), FGF7 (KGF), FGF9, FGF1 0, FGF 11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FELL (EPSELON), FEL1 (ZETA), IL 1A, IL 1B, IL2, IL3, IL4, IL5, IL6, IL7, IL8, IL9, IL1 0, IL 11, IL 12A, IL 12B, IL 13, IL 14, IL 15, IL 16, IL 17, IL 17B, IL 18, IL 19, 1L20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNF SF10 (TRAIL), TNFSF11 (TRANCE), TNFSF12 (AP03L), TNFSF13 (April), TNFSF13B, TNF SF14 (HVEM-L), TNF SF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 1IRA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6 ST, T, IL18BP, IL18RAP, IL22RA2, AIF1, HGF, LEP (leptin), PTN, and THPO.k

In certain embodiments, an antibody produced by cells and methods disclosed herein is capable of binding to a chemokine, chemokine receptor, or a chemokine-related protein selected from the group consisting of CCLI (1-309), CCL2 (MCP-1/1VICAF), CCL3 (MIP-Iα), CCL4 (MIP-1p), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-I(3), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8RP), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1a, DL8, PRL, RGS3, RGS13, SDF2, SLIT2, TLR2, TLR4, TREM1, TREM2, and VHL.

In certain embodiments, an antibody produced by methods disclosed herein (e.g., a multispecific antibody such as a bispecific antibody) is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3 p); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Ia); CCL4 (MDP-Iβ); CCL5 (RANTES); CCL7 (MCP-3); CCL8 (mcp-2); CCNA1; CCNA2; CCND1; CCNE1; CCNE2; CCR1 (CKRI/HM145); CCR2 (mcp-IR(3/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FELL (EPSILON); FILL (ZETA); FLJ12584; FLJ25530; FLRTI (fibronectin); FLT1; FOS; FOSL1 (FRA-1); FY (DARC); GABRP (GABAa); GAGEBI; GAGECI; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOPI; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF 1R; IGF2; IGFBP2; IGFBP3; IGFBP6; IL-1; 1L10; 1L10RA; IL10RB; 1L11; IL11RA; IL-12; IL12A; IL12B; IL12RB1; IL12RB2; IL13; IL13RA1; IL13RA2; 1L14; IL15; IL15RA; IL16; IL17; IL17B; IL17C; IL17R; IL18; IL18BP; IL18R1; IL18RAP; IL19; ILIA; IL1B; ILIF10; IL1F5; IL1F6; IL1F7; IL1F8; IL1F9; IL1HYL IL1R1; IL1R2; IL1RAP; LL1RAPL1; IL1RAPL2; IL1RLL IL1RL2, IL1RN; IL2; IL20; IL20Ra; IL21 R; IL22; IL-22c; IL22R; IL22RA2; IL23; 11,24; IL25; IL26; IL27; IL28A; IL28B; IL29; IL2RA; IL2RB; IL2RG; IL3; 1L30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); a4r37 and ctE, f37 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67, RIG-E, SCA-2, TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NR0B2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3 C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PM; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARE; RET (ret proto-oncogene; MEN2A; HSCR1; MEN2B; MTC1; PTC; CDHF12; Hs 0.168114; RET51; RET-ELE1); RGSI; RGS13; RGS3; RNF 110 (ZNF 144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B); SEMINA1; SERPINA3; SERP1NB5 (maspin); SERPINE1 (PAI-1); SERPDMF 1; SHBG; SLA2; SLC2A2; SLC33A1; SLC43A1; SLIT2; SPPI; SPRR1B (Spr1); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSF HA; TNFRSF1A; TNFRSF1B; TNFRSF 21; TNFRSF 5; TNFRSF6 (Fas); TNFRSF7; TNFRSF8; TNFRSF9; TNFSF10 (TRAIL); TNFSF11 (TRANCE); TNF SF12 (AP03L); TNF SF13 (April); TNF SF13B; TNF SF 14 (HVEM-L); TNF SF 15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCAIA; tyrosinase; SHEP3); VEGF; VEGFB; VEGFC; versican; VHL C5; VLA-4; XCL1 (lymphotactin); XCL2 (SCM-1b); XCRI (GPR5/CCXCRI); YY1; and ZFPM2.

In certain embodiments, an antibody produced by the cells and methods disclosed herein is capable of binding to CD proteins such as CD3, CD4, CD5, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD79f3, IGb (immunoglobulin-associated beta), B29); CD200 members of the ErbB receptor family such as the EGF receptor, HER2, HER3, or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD1 1 a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mp1 receptor; CTLA-4; RANKL, RANK, RSV F protein, protein C etc.

In certain embodiments, the cells and methods provided herein can be used to produce an antibody (or a multispecific antibody, such as a bispecific antibody) that specifically binds to complement protein C5 (e.g., an anti-05 agonist antibody that specifically binds to human C5). In certain embodiments, the anti-05 antibody comprises 1, 2, 3, 4, 5 or 6 CDRs selected from (a) a heavy chain variable region CDR1 comprising the amino acid sequence of SSYYMA (SEQ ID NO: 1); (b) a heavy chain variable region CDR2 comprising the amino acid sequence of AIFTGSGAEYKAEWAKG (SEQ ID NO:26); (c) a heavy chain variable region CDR3 comprising the amino acid sequence of DAGYDYPTHAMHY (SEQ ID NO: 27); (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). For example, in certain embodiments, the anti-05 antibody comprises a heavy chain variable domain (VH) sequence comprising one, two or three CDRs selected from: (a) a heavy chain variable region CDR1 comprising the amino acid sequence of (SSYYMA (SEQ ID NO: 1); (b) a heavy chain variable region CDR2 comprising the amino acid sequence of AIFTGSGAEYKAEWAKG (SEQ ID NO: 26); (c) a heavy chain variable region CDR3 comprising the amino acid sequence of DAGYDYPTHAMHY (SEQ ID NO: 27); and/or a light chain variable domain (VL) sequence comprising one, two or three CDRs selected from (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). The sequences of CDR1, CDR2 and CDR3 of the heavy chain variable region and CDR1, CDR2 and CDR3 of the light chain variable region above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.)

In certain embodiments, the anti-05 antibody comprises the VH and VL sequences

QVQLVESGGG LVQPGRSLRL SCAASGFTVH SSYYMAWVRQ APGKGLEWVG AIFTGSGAEY KAEWAKGRVT ISKDTSKNQV VLTMTNMDPV DTATYYCASD AGYDYPTHAM HYWGQGTLVT VSS (SEQ ID NO: 31)

and

DIQMTQSPSS LSASVGDRVT ITCRASQGIS SSLAWYQQKP GKAPKLLIYG ASETESGVPS RFSGSGSGTD FTLTISSLQP EDFATYYCQN TKVGSSYGNT FGGGTKVEIK (SEQ ID NO: 32), respectively, including post-translational modifications of those sequences. The VH and VL sequences above are disclosed in US 2016/0176954 as SEQ ID NO: 106 and SEQ ID NO: 111, respectively. (See Tables 7 and 8 in US 2016/0176954.) In certain embodiments, the anti-05 antibody is 305L015 (see US 2016/0176954).

In certain embodiments, an antibody produced by methods disclosed herein is capable of binding to 0X40 (e.g., an anti-0X40 agonist antibody that specifically binds to human 0X40). In certain embodiments, the anti-0X40 antibody comprises 1, 2, 3, 4, 5 or 6 CDRs selected from (a) a heavy chain variable region CDR1 comprising the amino acid sequence of DSYNIS (SEQ ID NO: 2); (b) a heavy chain variable region CDR2 comprising the amino acid sequence of DMYPDNGDSSYNQKFRE (SEQ ID NO: 3); (c) a heavy chain variable region CDR3 comprising the amino acid sequence of APRWYFSV (SEQ ID NO: 4); (d) a light chain variable region CDR1 comprising the amino acid sequence of RASQDISNYLN (SEQ ID NO: 5); (e) a light chain variable region CDR2 comprising the amino acid sequence of YTSRLRS (SEQ ID NO: 6); and (f) a light chain variable region CDR3 comprising the amino acid sequence of QQGHTLPPT (SEQ ID NO: 7). For example, in certain embodiments, the anti-OX40 antibody comprises a heavy chain variable domain (VH) sequence comprising one, two or three CDRs selected from: (a) a heavy chain variable region CDR1 comprising the amino acid sequence of DSYNIS (SEQ ID NO: 2); (b) a heavy chain variable region CDR2 comprising the amino acid sequence of DMYPDNGDSSYNQKFRE (SEQ ID NO: 3); and (c) a heavy chain variable region CDR3 comprising the amino acid sequence of APRWYFSV (SEQ ID NO: 4) and/or a light chain variable domain (VL) sequence comprising one, two or three CDRs selected from (a) a light chain variable region CDR1 comprising the amino acid sequence of RASQDISNYLN (SEQ ID NO: 5); (b) a light chain variable region CDR2 comprising the amino acid sequence of YTSRLRS (SEQ ID NO: 6); and (c) a light chain variable region CDR3 comprising the amino acid sequence of QQGHTLPPT (SEQ ID NO: 7). In certain embodiments, the anti-OX40 antibody comprises the VH and VL sequences

EVQLVQSGAE VKKPGASVKV SCKASGYTFT DSYMSWVRQA PGQGLEWIGD MYPDNGDSSY NQKFRERVTI TRDTSTSTAY LELSSLRSED TAVYYCVLAP RWYFSVWGQG TLVTVSS (SEQ ID NO: 8)

and

DIQMTQSPSS LSASVGDRVT ITCRASQDIS NYLNWYQQKP GKAPKLLIYY TSRLRSGVPS RFSGSGSGTD FTLTISSLQP EDFATYYCQQ GHTLPPTFGQ GTKVEIK (SEQ ID NO: 9), respectively, including post-translational modifications of those sequences.

In certain embodiments, the anti-0X40 antibody comprises 1, 2, 3, 4, 5 or 6 CDRs selected from (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); (c) a heavy chain variable region CDR3 comprising the amino acid sequence of DRLDY (SEQ ID NO: 12); (d) a light chain variable region CDR1 comprising the amino acid sequence of HASQDISSYIV (SEQ ID NO: 13); (e) a light chain variable region CDR2 comprising the amino acid sequence of HGTNLED (SEQ ID NO: 14); and (f) a light chain variable region CDR3 comprising the amino acid sequence of VHYAQFPYT (SEQ ID NO: 15). For example, in certain embodiments, the anti-0X40 antibody comprises a heavy chain variable domain (VH) sequence comprising one, two or three CDRs selected from: (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); and (c) a heavy chain variable region CDR3 comprising the amino acid sequence of DRLDY (SEQ ID NO: 12) and/or a light chain variable domain (VL) sequence comprising one, two or three CDRs selected from (a) a light chain variable region CDR1 comprising the amino acid sequence of HASQDISSYIV (SEQ ID NO: 13); (b) a light chain variable region CDR2 comprising the amino acid sequence of HGTNLED (SEQ ID NO: 14); and (c) a light chain variable region CDR3 comprising the amino acid sequence of VHYAQFPYT (SEQ ID NO: 15). In certain embodiments, the anti-OX40 antibody comprises the VH and VL sequences

EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEQ ID NO: 16)

and

DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SEQ ID NO: 17), respectively, including post-translational modifications of those sequences.

Further details regarding anti-0X40 antibodies are provided in WO 2015/153513, which is incorporated herein by reference in its entirety.

In certain embodiments, an antibody produced by the cells and methods disclosed herein is capable of binding to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain embodiments, an antibody produced by the cells and methods disclosed herein is capable of binding to low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain embodiments, an antibody produced by the cells and methods disclosed herein is a human IgG2 antibody against CD40. In certain embodiments, the anti-CD40 antibody is RG7876.

In certain embodiments, the cells and methods of the present disclosure can be used to product a polypeptide. For example, but not by way of limitation, the polypeptide is a targeted immunocytokine. In certain embodiments, the targeted immunocytokine is a CEA-IL2v immunocytokine. In certain embodiments, the CEA-IL2v immunocytokine is RG7813. In certain embodiments, the targeted immunocytokine is a FAP-IL2v immunocytokine. In certain embodiments, the FAP-IL2v immunocytokine is RG7461.

In certain embodiments, the multispecific antibody (such as a bispecific antibody) produced by the cells or methods provided herein is capable of binding to CEA and at least one additional target molecule. In certain embodiments, the multispecific antibody (such as a bispecific antibody) produced according to methods provided herein is capable of binding to a tumor targeted cytokine and at least one additional target molecule. In certain embodiments, the multispecific antibody (such as a bispecific antibody) produced according to methods provided herein is fused to IL2v (i.e., an interleukin 2 variant) and binds an ILI-based immunocytokine and at least one additional target molecule. In certain embodiments, the multispecific antibody (such as a bispecific antibody) produced according to methods provided herein is a T-cell bispecific antibody (i.e., a bispecific T-cell engager or BiTE).

In certain embodiments, the multispecific antibody (such as a bispecific antibody) produced according to methods provided herein is capable of binding to at least two target molecules selected from: IL-1 alpha and IL-1 beta, IL-12 and IL-1S; IL-13 and IL-9; IL-13 and IL-4; IL-13 and IL-5; IL-5 and IL-4; IL-13 and IL-1beta; IL-13 and IL-25; IL-13 and TARC; IL-13 and MDC; IL-13 and MEF; IL-13 and TGF--; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF, TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2, VEGF-A and VEGF-C, VEGF-C and VEGF-D, HER2 and DR5, VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, 1L4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

In certain embodiments, the multispecific antibody (such as a bispecific antibody) produced according to methods provided herein is an anti-CEA/anti-CD3 bispecific antibody. In certain embodiments, the anti-CEA/anti-CD3 bispecific antibody is RG7802. In certain embodiments, the anti-CEA/anti-CD3 bispecific antibody comprises the amino acid sequences set forth in SEQ ID NOs: 18-21 are provided below:

(SEQ ID NO: 18)
DIQMTQSPSS LSASVGDRVT ITCKASAAVG TYVAWYQQKP GKAPKLLIYS
ASYRKRGVPS RFSGSGSGTD FTLTISSLQP EDFATYYCHQ YYTYPLFTFG
QGTKLEIKRT VAAPSVEIFP PSDEQLKSGT ASVVCLLNNE YPREAKVQWK
VDNALQSGNS QESVTEQDSK DSTYSLSSTL TLSKADYEKH KVYACEVTHQ
GLSSPVTKSF NRGEC
(SEQ ID NO: 19)
QAVVTQEPSL TVSPGGTVTL TCGSSTGAVT TSNYANWVQE KPGQAFRGLI
GGTNKRAPGT PARFSGSLLG GKAALTLSGA QPEDEAEYYC ALWYSNLWVE
GGGTKLTVLS SASTKGPSVF PLAPSSKSTS GGTAALGCLV KDYFPEPVTV
SWNSGALTSG VHTFPAVLQS SGLYSLSSVV TVPSSSLGTQ TYICNVNHKP
SNTKVDKKVE PKSC
(SEQ ID NO: 20)
QVQLVQSGAE VKKPGASVKV SCKASGYTFT EFGMNWVRQA PGQGLEWMGW
INTKTGEATY VEEFKGRVTF TTDTSTSTAY MELRSLRSDD TAVYYCARWD
FAYYVEAMDY WGQGTTVTVS SASTKGPSVE PLAPSSKSTS GGTAALGCLV
KDYFPEPVTV SWNSGALTSG VHTFPAVLQS SGLYSLSSVV TVPSSSLGTQ
TYICNVNHKP SNTKVDKKVE PKSCDGGGGS GGGGSEVQLL ESGGGLVQPG
GSLRLSCAAS GFTFSTYAMN WVRQAPGKGL EWVSRIRSKY NNYATYYADS
VKGRFTISRD DSKNTLYLQM NSLRAEDTAV YYCVRHGNFG NSYVSWFAYW
GQGTLVTVSS ASVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SFNRGECDKT HTCPPCPAPE AAGGPSVFLF PPKPKDTLMI
SRTPEVTCVV VDVSHEDPEV KFNWYVDGVE VHNAKTKPRE EQYNSTYRVV
SVLTVLHQDW LNGKEYKCKV SNKALGAPIE KTISKAKGQP REPQVYTLPP
CRDELTKNQV SLWCLVKGFY PSDIAVEWES NGQPENNYKT TPPVLDSDGS
FFLYSKLTVD KSRWQQGNVF SCSVMHEALH NHYTQKSLSL SPGK
(SEQ ID NO: 21)
QVQLVQSGAE VKKPGASVKV SCKASGYTFT EFGMNWVRQA PGQGLEWMGW
WINTKTGEATY VEEFKGRVTF TTDTSTSTAY MELRSLRSDD TAVYYCARWD
FAYYVEAMDY YWGQGTTVTVS SASTKGPSVE PLAPSSKSTS GGTAALGCLV
KDYFPEPVTV SWNSGALTSG GVHTFPAVLQS SGLYSLSSVV TVPSSSLGTQ
TYICNVNHKP SNTKVDKKVE PKSCDKTHT CPPCPAPEAAG GPSVFLFPPK
PKDTLMISRT PEVTCVVVDV SHEDPEVKFN WYVDGVEVH NAKTKPREEQY
NSTYRVVSVL TVLHQDWING KEYKCKVSNK ALGAPIEKTI SKAKGQPRE
PQVCTLPPSRD ELTKNQVSLS CAVKGFYPSD IAVEWESNGQ PENNYKTTPP
VLDSDGSFF LVSKLTVDKSR WQQGNVFSCS VMHEALHNHY TQKSLSLSPG K

Further details regarding anti-CEA/anti-CD3 bispecific antibodies are provided in WO 2014/121712, which is incorporated herein by reference in its entirety.

In certain embodiments, a multispecific antibody (such as a bispecific antibody) produced by the cells and methods disclosed herein is an anti-VEGF/anti-angiopoietin bispecific antibody. In certain embodiments, the anti-VEGF/anti-angiopoietin bispecific antibody bispecific antibody is a Crossmab. In certain embodiments, the anti-VEGF/anti-angiopoietin bispecific antibody is RG7716. In certain embodiments, the anti-CEA/anti-CD3 bispecific antibody comprises the amino acid sequences set forth in SEQ ID NOs: 22-25 are provided below.

(SEQ ID NO: 22)
EVQLVESGGG LVQPGGSLRL SCAASGYDET HYGMNWVRQA PGKGLEWVGW
INTYTGEPTY AADEKRRFTF SLDTSKSTAY LQMNSLRAED TAVYYCAKYP
YYYGTSHWYF DVWGQGTLVT VSSASTKGPS VFFLAPSSKS TSGGTAALGC
LVKDYFFEPV TVSWNSGALT SGVHTFFAVL QSSGLYSLSS VVTVPSSSLG
TQTYICNVNH KPSNTKVDKK VEPKSCDKTH TCPPCPAPEA AGGPSVFLFP
PKPKDTLMAS RTPEVTCVVV DVSHEDPEVK FNWYVDGVEV HNAKTKPREE
QYNSTYRVVS VLTVLAQDWL NGKEYKCKVS NKALGAPIEK TISKAKGQPR
EPQVYTLPPC RDELTKNQVS LWCLVKGFYP SDIAVEWESN GQPENNYKTT
PPVLDSDGSF FLYSKLTVDK SRWQQGNVFS CSVMHEALHN AYTQKSLSLS PGK
(SEQ ID NO: 23)
QVQLVQSGAE VKKPGASVKV SCKASGYTFT GYYMHWVRQA PGQGLEWMGW
INPNSGGTNY AQKFQGRVTM TRDTSISTAY MELSRLRSDD TAVYYCARSP
NPYYYDSSGY YYPGAFDIWG QGTMVTVSSA SVAAPSVFIF PPSDEQLKSG
TASVVCLLNN FYPREAKVQW KVDNALQSGN SQESVTEQDS KDSTYSLSST
LTLSKADYEK HKVYACEVTH QGLSSPVTKS FNRGECDKTH TCPPCPAPEA
AGGPSVFLFP PKPKDTLMAS RTPEVTCVVV DVSHEDPEVK FNWYVDGVEV
HNAKTKPREE QYNSTYRVVS VLTVLAQDWL NGKEYKCKVS NKALGAPIEK
TISKAKGQPR EPQVCTLPPS RDELTKNQVS LSCAVKGFYP SDIAVEWESN
GQPENNYKTT PPVLDSDGSF FLVSKLTVDK SRWQQGNVFS CSVMHEALHN
AYTQKSLSLS PGK
(SEQ ID NO: 24)
DIQLTQSFSS LSASVGDRVT ITCSASQDIS NYLNWYQQKP GKAPKVLIYF
TSSLHSGVPS RFSGSGSGTD FTLTISSLQP EDFATYYCQQ YSTVPWTFGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC
(SEQ ID NO: 25)
SYVLTQPPSV SVAPGQTARI TCGGNNIGSK SVHWYQQKPG QAPVLVVYDD
SDRPSGIPER FSGSNSGNTA TLTISRVEAG DEADYYCQVW DSSSDHWVFG
GGTKLTVLSS ASTKGPSVFP LAPSSKSTSG GTAALGCLVK DYFPEPVTVS
WNSGALTSGV HTFPAVLQSS GLYSLSSVVT VPSSSLGTQT YICNVNHKPS
NTKVDKKVEP KSC

In certain embodiments, the multispecific antibody (such as a bispecific antibody) produced by methods disclosed herein is an anti-Ang²/ₐnti-VEGF bispecific antibody. In certain embodiments, the anti-Ang²/ₐnti-VEGF bispecific antibody is RG7221. In certain embodiments, the anti-Ang²/ₐnti-VEGF bispecific antibody is CAS Number 1448221-05-3.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or can be cells which have been transformed by recombinant techniques to express the transmembrane molecule. Other antigens and forms thereof useful for preparing antibodies will be apparent to those in the art.

In certain embodiments, the polypeptide (e.g., antibodies) produced by the cells and methods disclosed herein is capable of binding to can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using the methods described herein can contain the cytotoxic agent conjugated to a constant region of only one of the heavy chains or only one of the light chains.

### 5.5.1.8 Antibody Variants

In certain aspects, amino acid sequence variants of the antibodies provided herein are contemplated, e.g., the antibodies provided in Section 5.5.5. For example, it can be desirable to alter the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### 5.5.1.9 Substitution, Insertion, and Deletion Variants

In certain aspects, antibody variants having one or more amino acid substitutions are provided. Sites of interest for substitutional mutagenesis include the CDRs Clean Copy of Substitute Specification Attorney Docket No. 001B206. 1392 and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) can be made in CDRs, e.g., to improve antibody affinity. Such alterations can be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling. CDR-H3 and CDR-L3 in particular are often targeted.

In certain aspects, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity can be made in the CDRs. Such alterations can, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### 5.5.1.10 Glycosylation Variants

In certain aspects, an antibody provided herein is altered to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the oligosaccharide attached thereto can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure can be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides can be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e.g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 can also be located about +3 amino acids upstream or downstream of position 297, i.e., between positions 294 and 300, due to minor sequence variations in antibodies. Such antibodies having an increased proportion of non-fucosylated oligosaccharides in the Fc region can have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants can have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants can have improved CDC function. Such antibody variants are described, e.g., in WO 1997/30087; WO 1998/58964; and WO 1999/22764.

### 5.5.1.11 Fe Region Variants

In certain aspects, one or more amino acid modifications can be introduced into the Fc region of an antibody provided herein, thereby generating an Fc region variant. The Fc region variant can comprise a human Fc region sequence (e.g., a human IgG1, IgG2, IgG3 or IgG4 Fc region) comprising an amino acid modification (e.g., a substitution) at one or more amino acid positions.

In certain aspects, the present disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTITM non-radioactive cytotoxicity assay for flow cytometry (CellTechnology, Inc. Mountain View, CA; and CytoTox 96@ non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Intl. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fe region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and 11435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 76677671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### 5.5.1.12 Cysteine Engineered Antibody Variants

In certain aspects, it can be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. Cysteine engineered antibodies can be generated as described, e.g., in U.S. Pat. Nos. 7,521,541, 8,30,930, 7,855,275, 9,000,130, or WO 2016040856.

### 5.5.1.13 Antibody Derivatives

In certain aspects, an antibody provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 5.5.1.14 Immunoconjugates

The present disclosure also provides immunoconjugates comprising an antibody disclosed herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it can comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### 5.6 Exemplary Non-Limiting Embodiments

A. A modified cell, wherein the cell is modified to reduce or eliminate the expression of two or more endogenous proteins relative to the expression of the endogenous proteins in an unmodified cell, wherein:
   (a) one or more of the endogenous proteins having reduced or eliminated expression promotes apoptosis of the modified cell during cell culture; and
   (b) one or more of the endogenous proteins having reduced or eliminated expression regulates the unfolded protein response (UPR).
B. A modified cell, wherein the cell is modified to reduce or eliminate the expression of two or more endogenous proteins relative to the expression of the endogenous proteins in an unmodified cell, wherein one or more endogenous proteins is selected from the endogenous protein group consisting of: BCL2 Associated X, Apoptosis Regulator (BAX); and BCL2 Antagonist/Killer 1 (BAK); and one of the endogenous proteins is Protein Kinase R-like ER Kinase (PERK).
B1. The modified cell of B, wherein the expression of BAX, BAK, and PERK is reduced or eliminated.
B2. The modified cell of any one of A-B1, wherein the modified cell is engineered to express a recombinant product of interest.
B3. The modified cell of any one of A-B1, wherein the modified cell is generated from a recombinant cell that expresses a recombinant product of interest.
B4. The modified cell of B2 or B3, wherein the one or more endogenous proteins have no detectable expression.
B5. The modified cell of B2 or B3, wherein the recombinant product of interest comprises a viral vector.
B6. The modified cell of B2 or B3, wherein the recombinant product of interest comprises a viral particle.
B7. The modified cell of B2 or B3, wherein the recombinant product of interest comprises a recombinant protein.
B8. The modified cell of B7, wherein the recombinant protein is antibody or an antibody-fusion protein or an antigen-binding fragment thereof.
B9. The modified cell of B8, wherein the antibody is a multispecific antibody or an antigen-binding fragment thereof.
B10. The modified cell of B8, wherein the antibody consists of a single heavy chain sequence and a single light chain sequence or antigen-binding fragments thereof.
B11. The modified cell of any one of B8-B10, wherein the antibody is a chimeric antibody, a human antibody or a humanized antibody.
B12. The modified cell of any one of B8-B11, wherein the antibody is a monoclonal antibody.
B13. The modified cell of B2 or B3, wherein the recombinant product of interest is encoded by an exogenous nucleic acid sequence integrated in the cellular genome at one or more targeted locations.
B14. The modified cell of any one of A-B13, wherein the modified cell does not express detectable BAX, BAK, and PERK.
B15. The modified cell of any one of A-B13, wherein the modified cell expresses decreased levels of BAX, BAK, and PERK.
B16. The modified cell of any one of A-B15, wherein the modified cell is a modified animal cell.
B17. The modified animal cell of B16, wherein the modified animal cell is a modified Sf9, CHO, HEK 293, HEK-293T, BHK, A549, or HeLa cell.
B18. A composition comprising the modified cell of any one of A-B17.
B19. A method of producing a recombinant product of interest comprising:
   (a) culturing a modified cell of any one of A-B17; and
   (b) recovering the recombinant product of interest from a cultivation medium or the modified cells, wherein the modified cells expressing the recombinant product of interest exhibit reduced or eliminated expression of BAX, BAK, and PERK.
C. A method for producing a modified cell, comprising:
   (a) applying a nuclease-assisted and/or nucleic acid targeting BAX, BAK, and PERK, in the cell to reduce or eliminate the expression of said endogenous genes, and
   (b) selecting the modified cell wherein the expression of said endogenous genes have been reduced or eliminated as compared to an unmodified cell.
C1. The method according to C, wherein the modification is performed before the introduction of the exogenous nucleic acid encoding the recombinant product of interest, or after the introduction of the exogenous nucleic acid encoding the recombinant product of interest.
C2. The method according to C or C1, wherein the nuclease-assisted gene targeting system is selected from the group consisting of CRISPR/Cas9, CRISPR/Cpf1, zinc-finger nuclease, TALEN or meganuclease.
C3. The method according to C or C1, wherein the reduction of gene expression is mediated by RNA silencing.
C4. The method according to C3, wherein RNA silencing is selected from the group consisting of siRNA gene targeting and knock-down, shRNA gene targeting and knock-down, and miRNA gene targeting and knock-down.
C5. The method of any one of B19 and C1-C4, wherein the recombinant product of interest is encoded by a nucleic acid sequence.
C6. The method of any one of B19 and C1-05, wherein the nucleic acid sequence is integrated in the cellular genome of the modified cells at one or more targeted locations.
C7. The method of any one of B19 and C1-05, wherein the recombinant product of interest expressed by the modified cells is encoded by a nucleic acid sequence that is randomly integrated in the cellular genome of the modified cells.
C8. The method of any one of B19 and C1-C7, wherein the recombinant product of interest comprises a viral vector.
C9. The method of any one of B19 and C1-C7, wherein the recombinant product of interest comprises a viral particle.
C10. The method of any one of B19 and C 1-C7, wherein the recombinant product of interest comprises a recombinant protein.
C11. The method of C10, wherein the recombinant protein is an antibody or an antibody-fusion protein or an antigen-binding fragment thereof.
C12. The method of C11, wherein antibody is a multispecific antibody or an antigen-binding fragment thereof.
C13. The method of C11, wherein the antibody consists of a single heavy chain sequence and a single light chain sequence or antigen-binding fragments thereof.
C14. The method of any one of C11-C13, wherein the antibody is a chimeric antibody, a human antibody or a humanized antibody.
C15. The method of any one of C11-C13, wherein the antibody is a monoclonal antibody.
C16. The method of any one of B19-C15 comprising purifying the product of interest, harvesting the product of interest, and/or formulating the product of interest.
C17. The method of C16, wherein the modified cell is a modified animal cell.
C18. The method of any one of B19-C17, wherein the modified animal cell is a modified Sf9, CHO, HEK 293, HEK 293T, BHK, A549, or HeLa cell.
C19. The modified cell or method of any of A-C18, wherein the modified cell has a higher specific productivity than a corresponding isolated animal cell that comprises the polynucleotide and functional copies of each of the wild type Bax, Bak, and PERK genes.
C20. The modified cell or method of any of A-C19, wherein the modified cell is more resistant to apoptosis than a corresponding isolated animal cell that comprises functional copies of each of the Bax, Bak, and PERK genes.
C21. The modified cell or method of any of A-C20, wherein the modified cell is employed in a fed-batch, perfusion, process intensified, semi-continuous perfusion, or continuous perfusion cell culture process.

### 6. EXAMPLES

The following examples are merely illustrative of the presently disclosed subject matter and should not be considered as limitations in any way.

### Materials and Methods

### Vector Constructs, Cell Culture Conditions and Production

Expression of both the heavy chain (HC) and light chain (LC), as two separate units, was directed by their respective cytomegalovirus (CMV) promoter and regulator elements. Plasmids encoded dihydrofolate reductase (DHFR) or puromycin as selection markers directed by the Simian Virus (SV) 40 early promoter and enhancer elements. The SV40 late polyadenylation (poly A) signal sequences were used in the 3′ region of the HC DNA and LC DNA. Cells were cultured in a proprietary serum-free DMEM/F12-based medium in 50-mL tube spin vessels shaking at 150 rpm, 37° C. and 5% C02 and were passaged at a seeding density of 4×10⁵ cells/mL every 3-4 days (Hu, et al., 2013).

Fed-batch production cultures were performed with proprietary chemically defined medium using different vessels (tube-spin and AMBR15) along with bolus feeds on Days 3, 7 and 10 as previously mentioned (Hsu, Aulakh, Traul, & Yuk, 2012). Anti-cell aggregation agent was used in all cultures during production assay to prevent cell aggregation due to the release of DNA from dying cells. Cells were seeded at low (1-2×10⁶ cells/mL) or high (10×10⁶ cells/mL) seeding densities using lean or rich production media. Cultures were temperature shifted from 37° C. to 35° C. on Day 3. Titers were determined using Protein-A affinity chromatography with UV detection. Percent viability and viable cell count were determined using a Vi-Cell XR instrument (Beckman Coulter Item #383721).

CRISPR/Cas9-mediated disruption of PERK (EIF2AK3)
sgRNA primer sequences were as follows:

PERK sgRNA 1: 5′ AGTCACGGCGGGCACTCGCG
PERK sgRNA 2: 5′ TACGGCCGAAGTGACCGTGG
PERK sgRNA 3: 5′ GCGTGACTCATGTTCGCCAG
Luciferase sgRNA: 5′ATCCTGTCCCTAGTGGCCC

Five million cells were washed and suspended in buffer R (Neon 100 uL kit cat: MPK 10025 Invitrogen). Five micrograms of Cas9:sgRNA RNP complex were added to the cell culture mixture. Cells were electroporated using 3×10 ms pulses at 1,620 V. The transfected cells were cultured for 3 days followed by single cell cloning via limiting dilution. Pool and single cell clones were screened for PERK knockout by western blot analysis.

RT-PCR analysis to detect IRE1alpha RNase activity

CHO-XBP1s Forward Primer: 5′ CCTTGTAATTGAGAACCAGG
CHO-XBP1s Reverse Primer: 5′CCAAAAGGATATCAGACTCGG

Power SYBR Green RNA-to CT-1 Step Kit and protocol used from Applied Biosystems (#4389986).

### Immunoblotting and Reagents

1.5 million cells were lysed in 1×NP40 buffer (10 mM Tris, pH 8.0, 0.5% NP40, 150 mM NaCl, 10 mM DTT and 5 mM MgC12) containing protease inhibitor cocktails (Roche EDTA free mini-tablets cocktail) for 20 min on ice. Lysates were subjected to sodium dodecyl sulfate-polyacrylamide gel electrophoresis (SDS-PAGE) (4-12% Tris glycine) and transferred to nitrocellulose membrane. After blocking with 5% milk in tris-buffered saline (TBS)-0.1% Tween buffer, the membranes were blotted with respective antibodies. Blots were visualized using HRP-conjugated anti-rabbit anti-body and SuperSignal West Dura Extended Duration Substrate. The following inhibitors were used: ATF6i (10 μM Ceapin-A7 (Gallagher, et al., 2016)), PERKi (10 μM Compound 39 (Axten, et al., 2012)), IRE1i6 (10 μM 4u8c (Cross, et al., 2012)), IRE1i9 (10 μM in-house/Genentech), PDGFRi (5-20 μM Abcam, AG-1296). The following antibodies were used: anti-PDGFRa (Cell Signaling Technology (CST), D1E1E), rabbit anti-BiP (C50B12, Cell Signaling Technology, 3177), rabbit anti-PERK (CST, C33E10), mouse anti-f3-actin-HRP (AC-15) (Abcam, ab49900), rabbit anti-phospho-Akt (Ser473) (CST, D9E), rabbit anti-Akt (CST, 5G3), rabbit cleaved caspase3 (CST, asp175), goat anti-human IgG-HRP (MP Biomedicals, 0855252), rabbit IRE1 a (CST, 14C10), mouse anti-phospho-IRE1, mouse anti-XBP1, rabbit anti-Bax (Abcam, ab32503), rabbit anti-Bak (CST, D4E4), donkey anti-rabbit IMP (Jackson ImmunoResearch Laboratories, Inc., 711 035-152), rabbit anti-sod2 (CST, D3X8F)

### Example 1: UPR Activation Attenuates PDGFRa Transcription and Downregulates its Expression

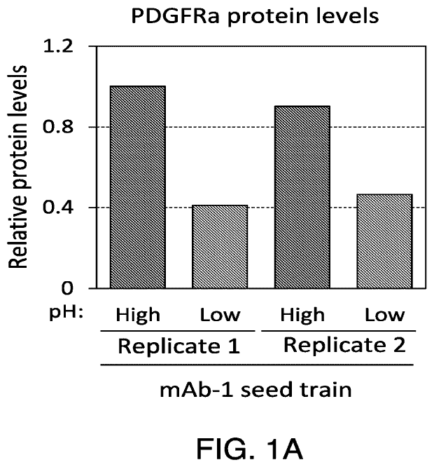

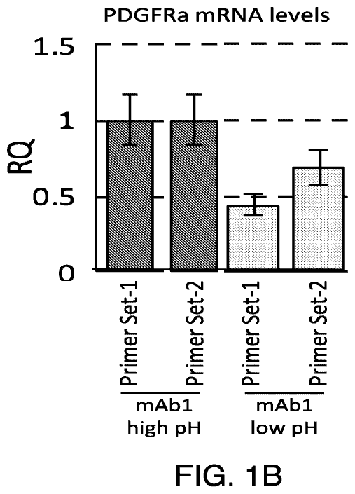

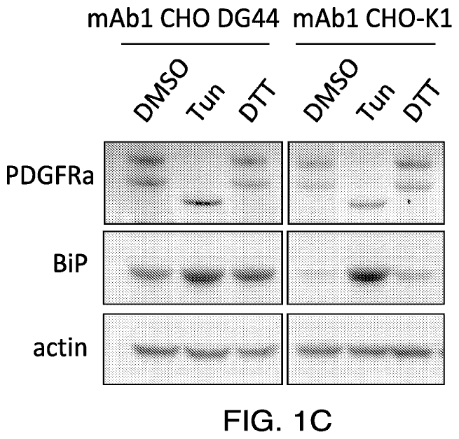

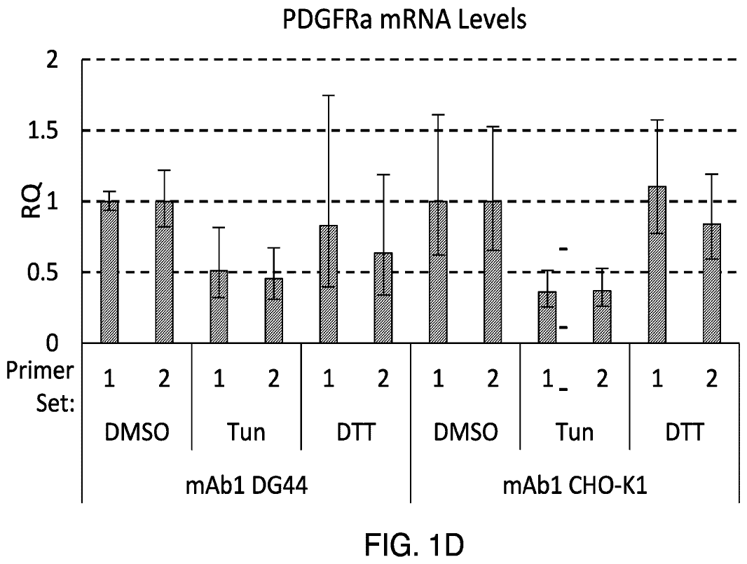

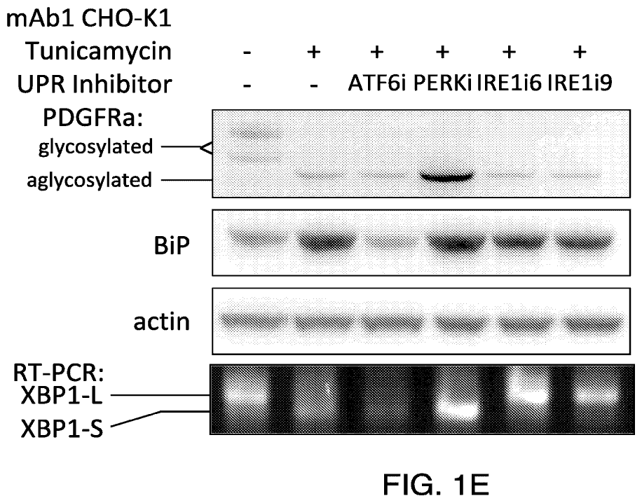

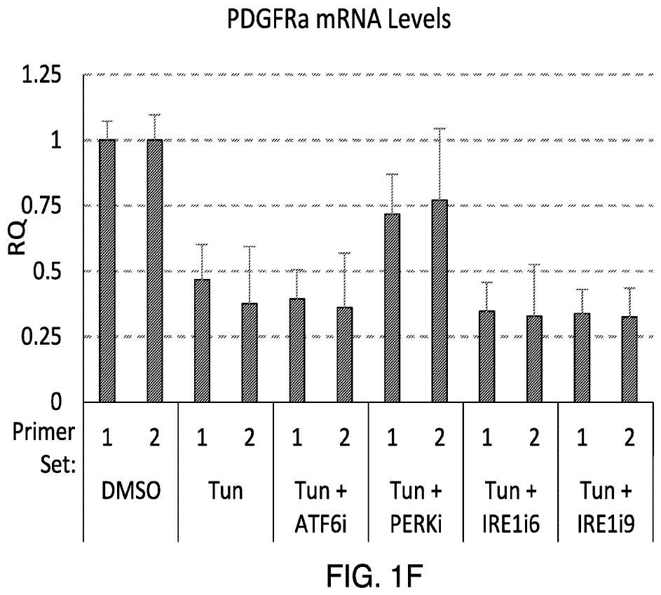

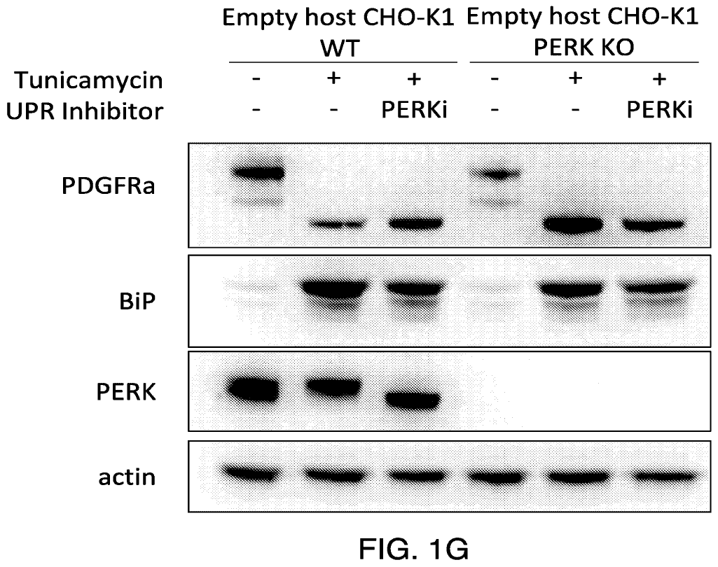

An interesting phenomenon was previously described where activation of UPR in seed train culture, triggered by lower pH conditions in a particular CHO cell line, negatively affected culture growth in production media at target pH (Tung, et al., 2018). When exposed to low pH conditions, high intracellular BiP levels were detected in this cell line that correlated to low growth profiles during production and poor bioprocess outcomes (Tung, et al., 2018). To better understand the underlying mechanism of reduced production culture growth in low vs high pH conditions, seed train cultures were maintained under high and low pH conditions and subjected to proteomic analysis by mass spectrometry. Significant reductions in expression of PDGFRa protein levels were observed under low pH conditions (FIG. 1A), which was linked to transcriptional attenuation of the PDGFRa gene (FIG. 1B). Since high intracellular BiP levels are indicative of UPR activation, it was decided to investigate the potential correlation between the UPR and reduced PDGFRa levels in CHO cells. Tunicamycin (Tun, strong UPR inducer) and DTT (weak UPR inducer) were used to chemically induce UPR in the seed train cultures of two antibody-expressing (mAb1) CHO host lines, CHO DG44 and CHO-K1. Under optimal pH conditions and with a strong UPR inducer (Tun), fully functional PDGFRa levels were reduced at both the protein and mRNA levels in both CHO host backgrounds (FIGS. 1C and 1D). Note that BiP levels, as an indicator of UPR activation, increase accordingly in response to strong and weak UPR chemical inducers (FIG. 1C). The lower molecular weight PDGFRa protein band observed upon tunicamycin treatment represents the non-glycosylated form of this protein as tunicamycin treatment inhibits protein glycosylation (FIG. 1C).

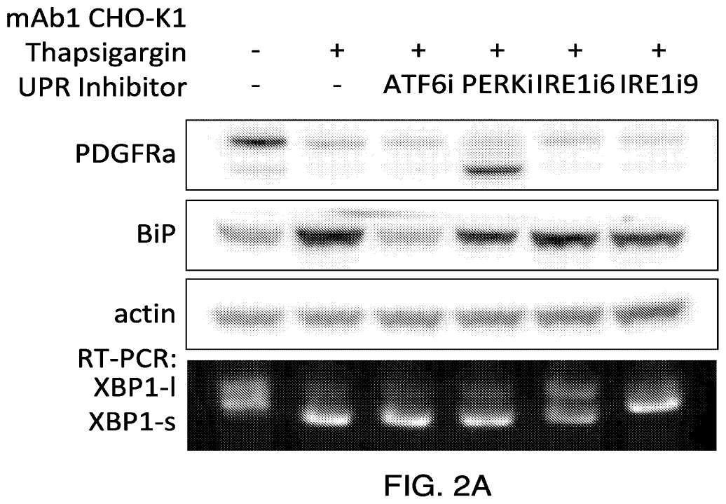

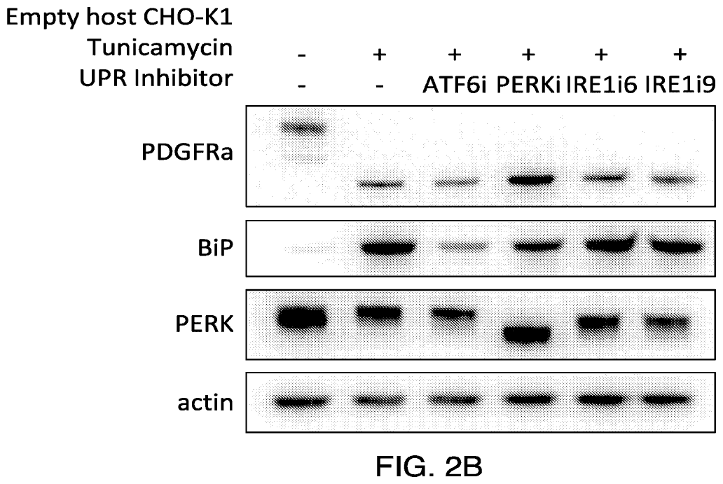

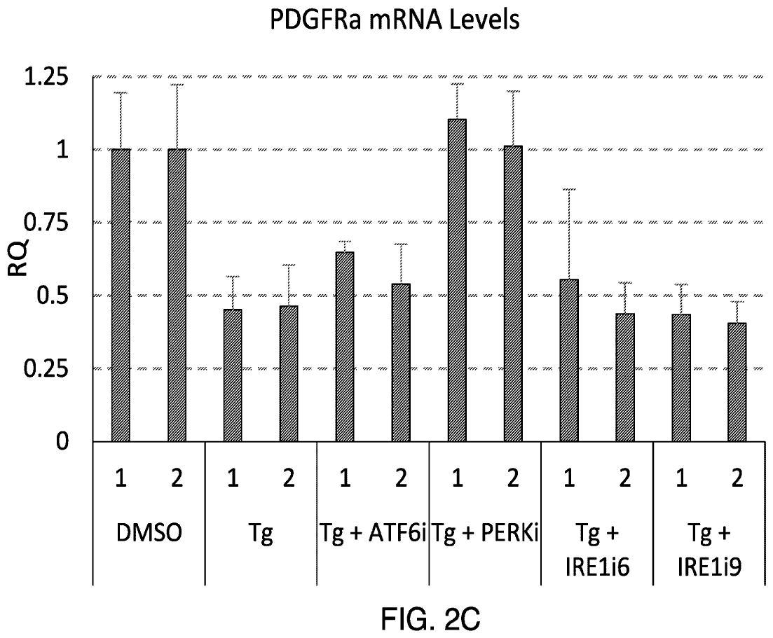

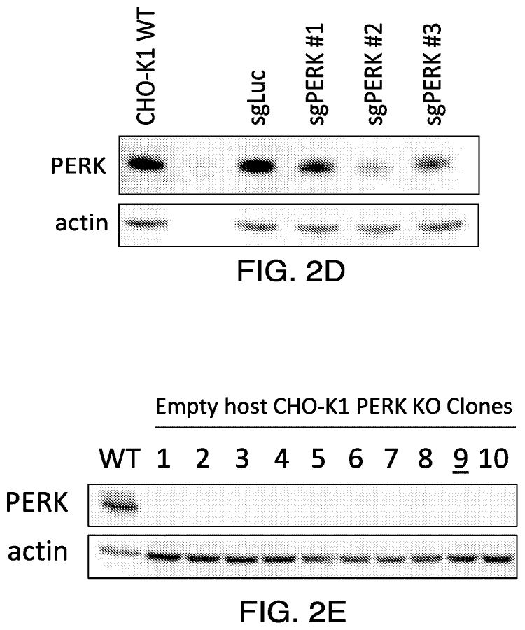

To further dissect which branch of the UPR is responsible for regulating PDGFRa levels, strong UPR inducers (tunicamycin and thapsigargin) were used to induce UPR in CHO-K1 cells treated with specific inhibitors against ATF6, PERK, or IRE1 a branches of the UPR pathway (FIGS. 1E, 1F and FIGS. 2A, 2B and 2C). These data revealed that inhibition of the PERK branch of the UPR pathway rescued downregulation of PDGFRa at the protein (FIG. 1E) and mRNA levels (FIG. 1F), without affecting activation of other branches of the UPR as evident by increased levels of intracellular BiP protein and XBP-1 RNA processing in both Tunicamycin (FIG. 1E) and Thapsigargin (FIG. 2A) treated cultures. Downregulation of PDGFRa via activation of the PERK branch of the UPR pathway occurred both in antibody-expressing (FIG. 1E and FIG. 2A) and empty host cells (FIG. 2B). The slightly lower molecular weight of PERK protein observed in the presence of PERK inhibitor is likely due to covalent modifications of PERK by this specific inhibitor (FIG. 2B).

Furthermore, sgRNAs were designed and tested to knockout the PERK gene in CHO-K1 cells using CRISPR-Cas9 (FIG. 2D) and a transfected a pool with the best knockout phenotype (sgPERK #2) was single cell cloned to isolate empty CHO-K1 host cell lines that did not express PERK protein (FIG. 2E). These empty CHO-K1 PERK KO host cell lines were evaluated for growth, transfection rate, recovery in selection media, and culture performance to identify a PERK KO host cell line with comparable overall culture performance to the wild-type (WT) CHO-K1 host. Empty WT and an empty PERK KO host cell line (Clone 9, FIG. 2E) were then treated with or without tunicamycin and PERK inhibitor to evaluate PDGFRa regulation upon UPR induction (FIG. 1G). Relative to the WT control, PDGFRa expression was not downregulated upon UPR induction and addition of PERK inhibitor did not further stabilize PDGFRa expression in the PERK KO host (FIG. 1G).

This investigation into one of our antibody-expressing cell lines, mAb 1 CHO DG44, revealed that transcriptional downregulation, and hence lower expression of PDGFRa protein, was the likely cause of poor growth outcomes during production when the cells were sourced from seed train cultures exposed to low pH (FIGS. 1A and 1B). It was previously shown that this poor growth outcome correlated with increased intracellular BiP levels, which is indicative of UPR activation (Tung, et al., 2018). When the UPR was chemically induced, PDGFRa protein levels also decreased due to transcriptional downregulation, a phenomenon that could be reversed by chemical inhibition of the PERK branch of the UPR pathway, suggesting that PERK activation mediates PDGFRa downregulation (FIGS. 1C, 1D, 1E, 1F, and FIGS. 2A, 2B and 2C). This was further confirmed when chemical induction of UPR in a PERK KO cell line did not result in downregulation of PDGFRa expression (FIG. 1G).

### Example 2: PDGFRa Signaling Pathway is Critical for CHO Culture Growth and Functions in Parallel to Insulin Signaling Pathway

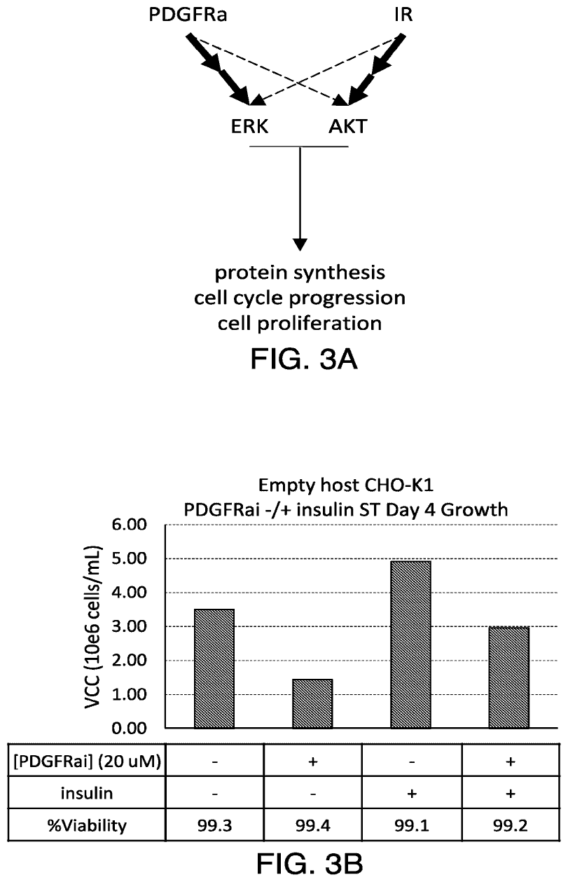

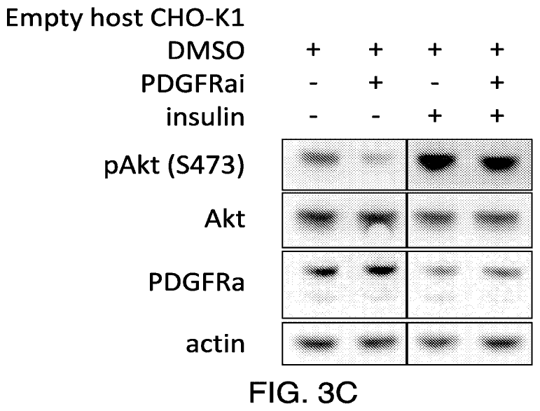

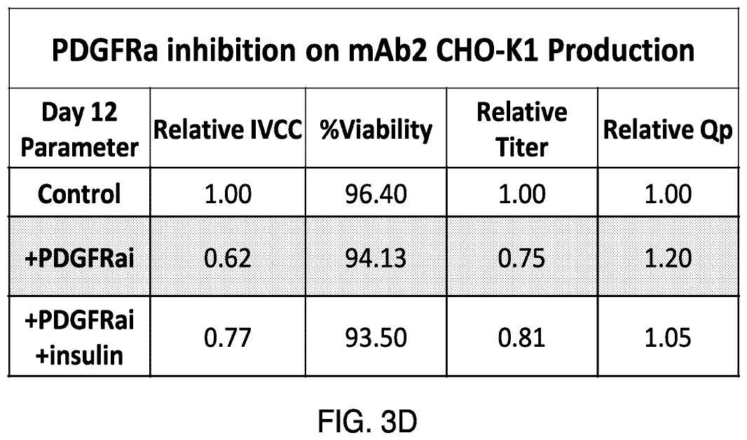

It was previously shown that UPR-induced poor growth profiles correlated with a decrease in PDGFRa levels (FIGS. 1A and 1B) (Tung, et al., 2018). PDGFRa and insulin signaling pathways have overlapping downstream targets (FIG. 3A), however insulin signaling negatively regulates PDGFRa signaling (Cirri, et al., 2005). To test the importance of PDGFRa signaling pathway in CHO cell growth, empty host CHO-K1 cells were cultured in the presence of different concentrations of PDGFRa inhibitor, which reduced cell growth by approximately 50% at 20 μM concentrations (FIG. 3B) due to attenuation of the Akt signaling pathway (FIG. 3C). Addition of insulin to the CHO cultures treated with the PDGFRa inhibitor partially rescued cellular growth (FIG. 3B) and increased Akt phosphorylation and hence its activation compared to the untreated cultures (FIG. 3C). These findings confirmed that PDGFRa and insulin signaling pathways indeed have overlapping downstream targets in CHO cells and that Akt signaling pathway remains intact in the presence of PDGFRa inhibitor (FIGS. 3B and 3C). PDGFRa signaling is also important for CHO production culture growth as its inhibition on Day 3 of a fed-batch production significantly decreased cell growth, without affecting cell viability, in an antibody-expressing (mAb2) CHO cell line (FIG. 3D). Similar to the seed train cultures (FIG. 3B), addition of insulin on Day 3 of the production culture partially rescued the observed cell growth inhibition (FIG. 3D).

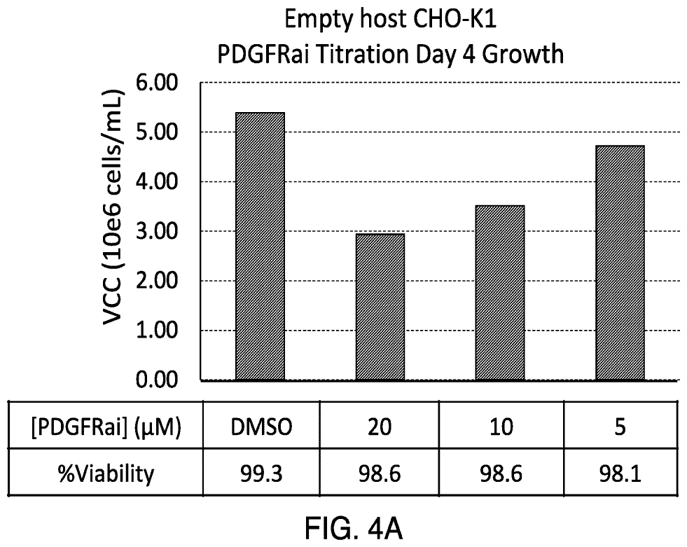

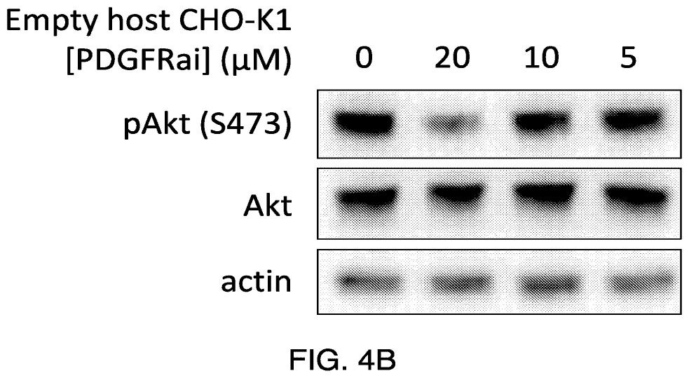

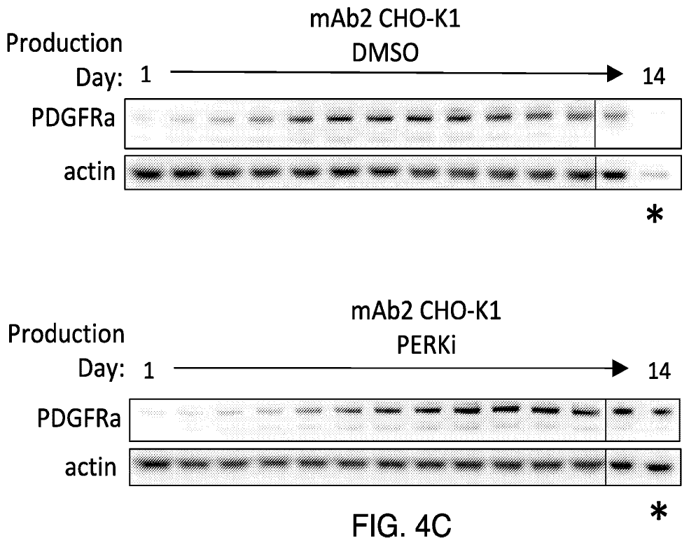

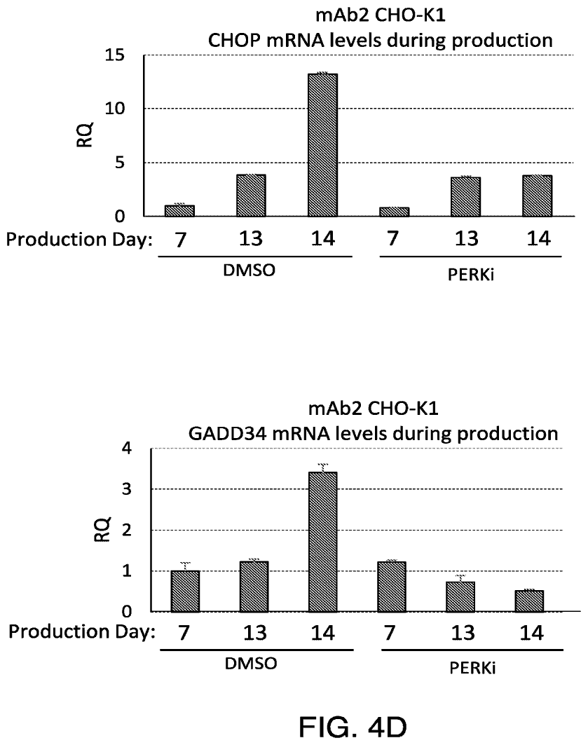

The PDGFRa signaling pathway proved to be critical for cell growth in our CHO cells, which are cultured in chemically defined media without any growth factors (FIGS. 4A and 4B), suggesting that either our CHO cells secrete a PDGFRa ligand, or PDGFRa signaling pathway is intrinsically active in these cells. Addition of insulin to the culture media partially rescued cell growth when PDGFRa signaling was inhibited, implicating that PDGFRa inhibitor is specific and does not affect downstream signaling (FIGS. 3B and 3C), as both PDGFRa and insulin receptor (IR) have partially overlapping signaling pathways (FIG. 3A).

Downregulation of PDGFRa by the PERK branch of the UPR was also observed in production culture where a decline in PDGFRa levels towards the end of the culture period, coincided with the higher levels of PERK activity as evident by a surge in mRNA levels of its downstream target proteins (FIGS. 4C and 4D). Chemical inhibition of PERK prevented transcriptional increase of its downstream targets and also stabilized PDGFRa levels during production (FIGS. 4C and 4D).

### Example 3: Activation of the PERK Branch of the UPR Attenuates PDGFRa Signaling, Reduces Specific Productivity and Promotes Culture Viability Durum Production

The correlation between PERK activation and downregulation of PDGFRa expression was monitored in production culture, using a mAb2-expressing CHO-K1 cell line, in the absence (control) or presence of PERK inhibitor (added on Day 3 of production). The observed downregulation of PDGFRa on days 13 and 14 of the production culture (FIG. 4C, left panel) correlated with an increase in mRNA levels of CHOP and GADD34 genes, which are downstream targets of PERK (Marciniak, et al., 2004), indicating activation of PERK signaling pathway (FIG. 4D). Addition of PERK inhibitor blocked PERK signaling (no increase in CHOP and GADD34 mRNA levels) and prevented downregulation of PDGFRa expression (FIGS. 4C right panel, and 4D). Since use of PERK inhibitor is cost prohibitive and its potential off-target activity on cultured cells cannot be fully ruled out, it was decided to generate PERK KO mAb2-expressing CHO-K1 cell lines to directly investigate the role of this signaling pathway in PDGFRa downregulation and production culture performance.

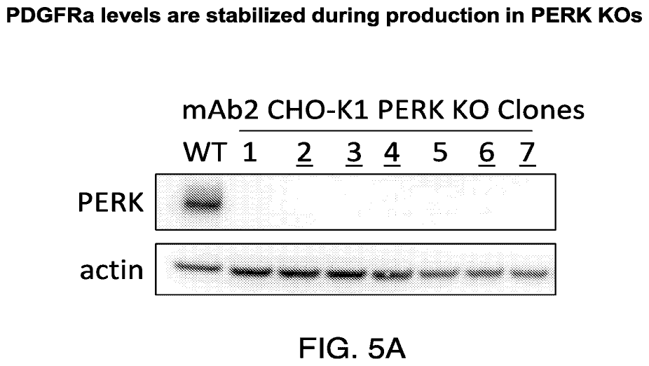

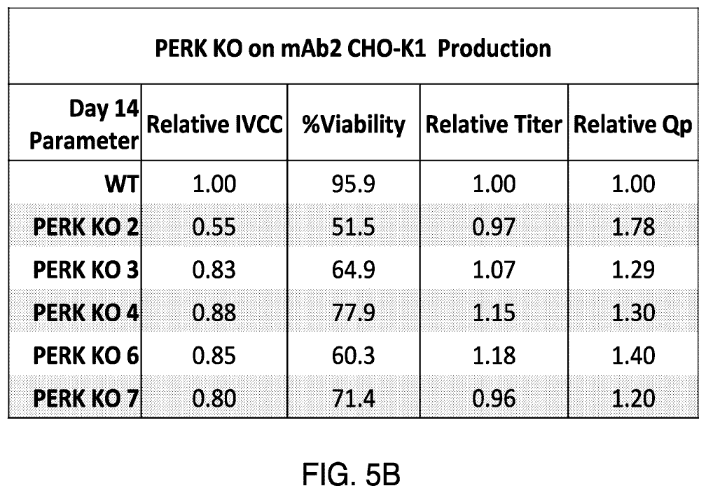

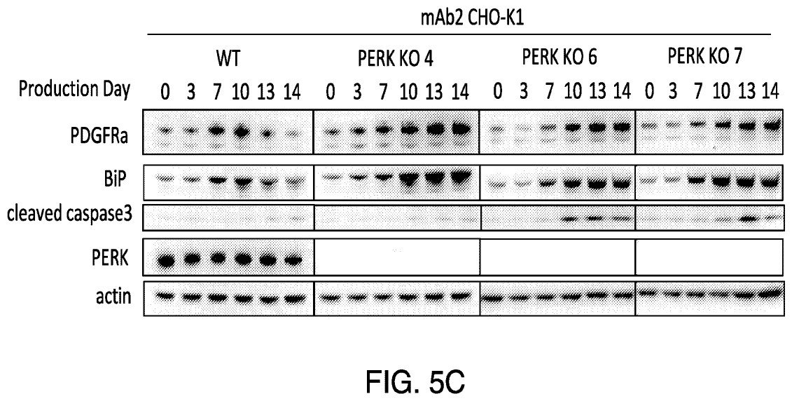

CRISPR-Cas9 technology was used to knockout the PERK gene in the mAb2-expressing CHO-K1 cell line and after single cell cloning, derived PERK KO cell lines with comparable growth profiles to the parental cell line (FIG. 5A, underlined clones) were evaluated in production culture (FIGS. 5B and 5C). PERK KO cell lines overall showed decreased growth and viability, compared to the parental cell line (FIG. 5B), however, all the PERK KO cell lines had higher specific productivities, and for most part titers, compared to the WT parental cell line (FIG. 5B). Western blot analysis of these cell lines during production confirmed that PDGFRa levels were stabilized in the PERK KO cell lines compared to the WT parental cell line, which displayed reduced levels of PDGFRa expression, towards the end of the production (FIG. 5C). Higher levels of intracellular BiP protein in the PERK KO cell lines indicated increased UPR activation (FIG. 5C), while the observed decrease in cellular growth and viability (FIG. 5B) correlated with increased caspase-3 cleavage, implying activation of the apoptotic pathway towards the end of the production culture (FIG. 5C). Antibodies expressed by WT or PERK KO cell lines had comparable product quality.

These findings confirmed that activation of the PERK branch of the UPR downregulates expression of PDGFRa in both seed train and production cultures. Interestingly, PERK KO cultures displayed lower overall viability and growth, but higher titer and specific productivity during production (FIG. 5B). Increased intracellular BiP levels and higher levels of caspase-3 cleavage in these cultures indicated activation of the UPR and apoptotic pathways, respectively, and correlated with lower culture viabilities (FIG. 5C). Likely, higher levels of specific productivity during production triggers cellular apoptosis and early PERK activation attenuates cellular apoptosis by simply reducing the specific productivity of these cells.

### Example 4: Knocking Out PERK in a Bax/Bak Double Knockout CHO Cell Line Drastically Increased Specific Productivity and Titer by Enhancing Transgene Transcription and Attenuating Apoptotic Cell Death

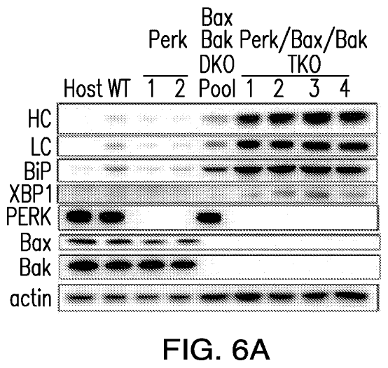

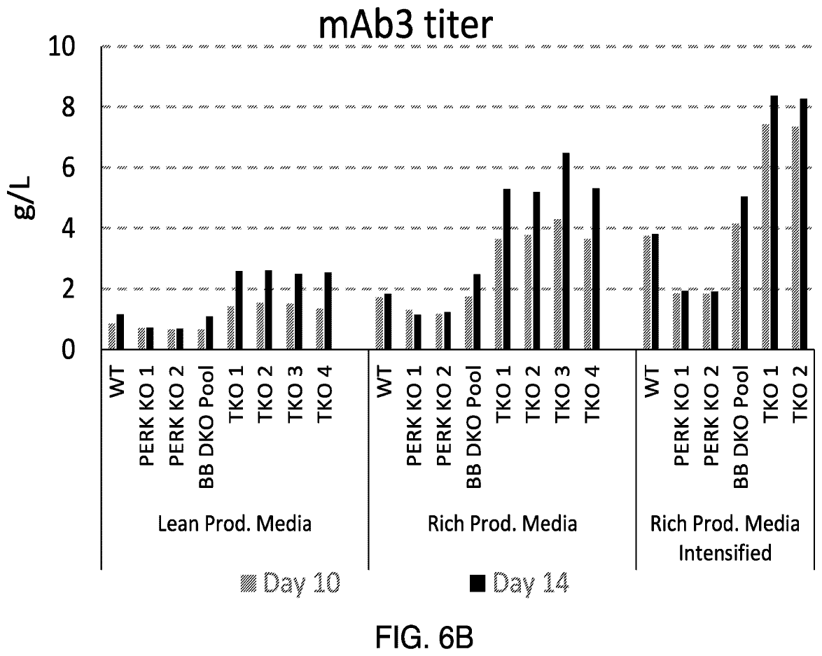

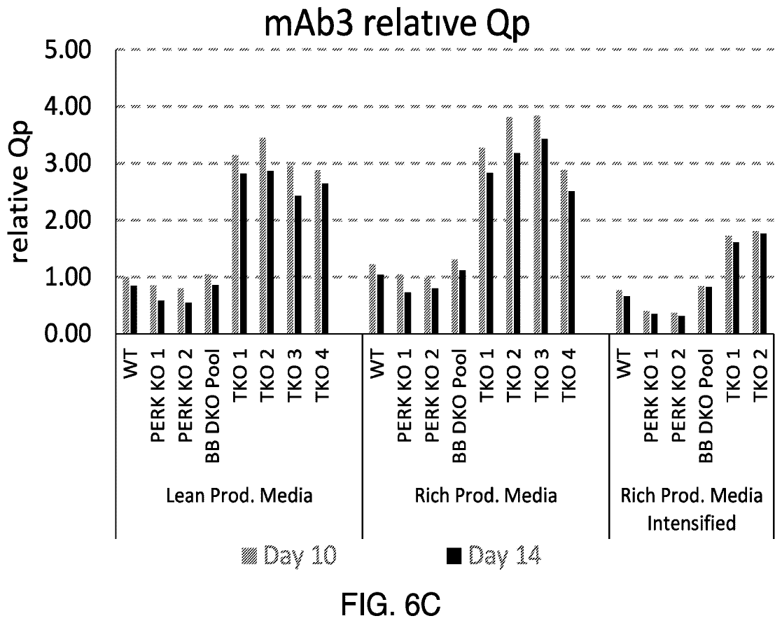

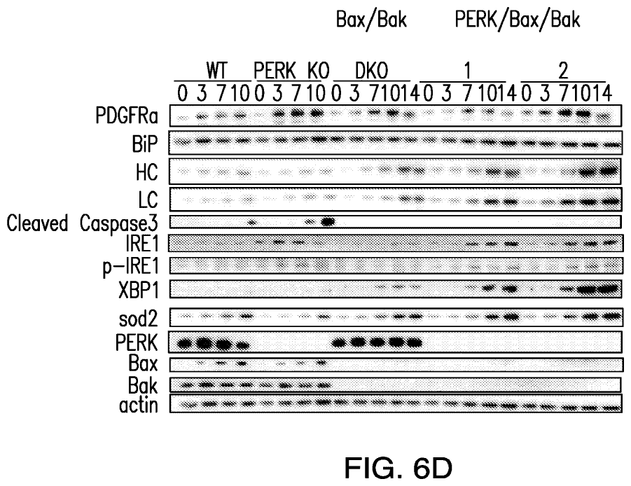

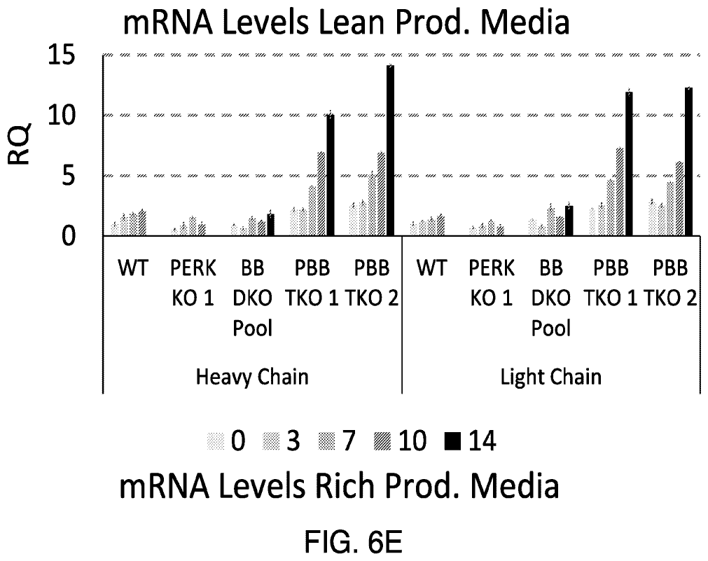

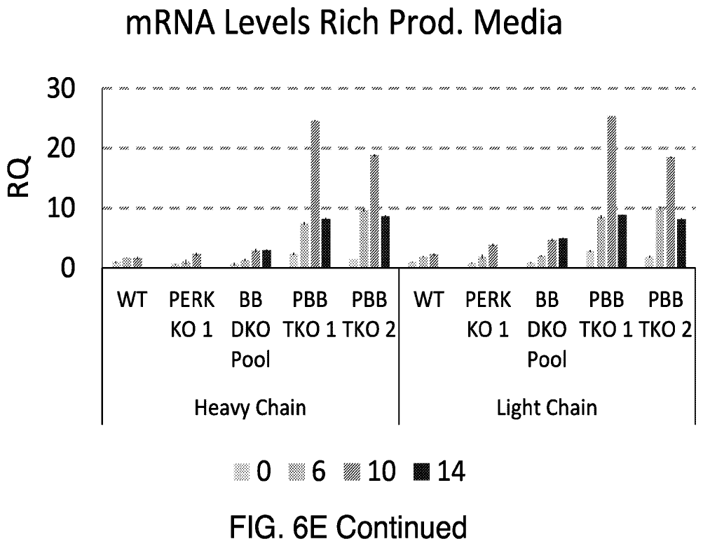

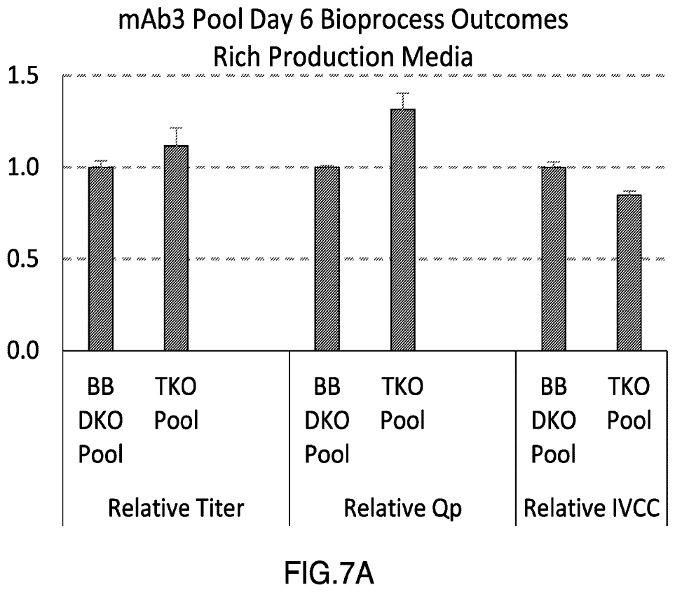

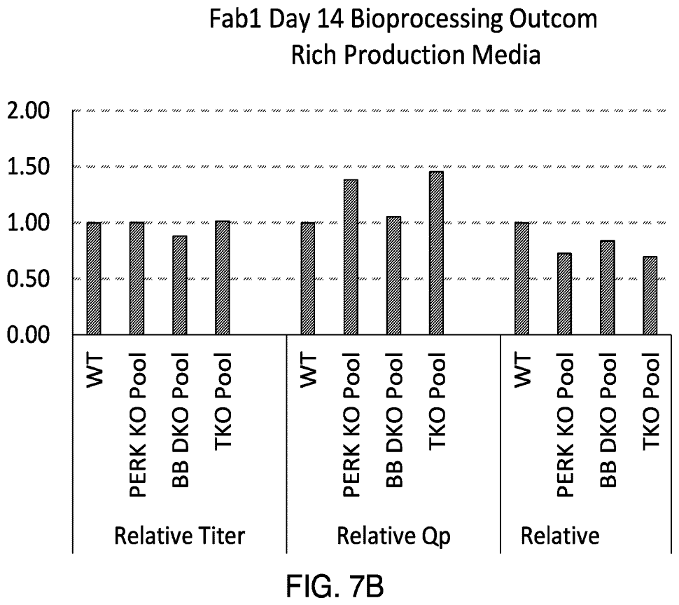

Since the PERK KO clones showed higher levels apoptosis during production (FIG. 5C), the PERK gene was knocked out in a mAb3-expressing WT cell line or a mAb3-expressing pool of Bax/Bak double knockout (DKO) cell line (FIG. 6A). Bax/Bak are proteins that act at the mitochondria to initiate apoptotic cell death (Taylor, Cullen, & Martin, 2008) and the deletion of these genes make cell lines more resistant to apoptosis and potentially improve viability and productivity during long production processes compared to WT CHO cell lines (Misaghi, Qu, Snowden, Chang, & Snedcor, 2013). After single cell sorting the PERK/Bax/Bak triple knockout (TKO) clones (FIG. 6A) were compared to controls (WT, PERK KO, and Bax/Bak DKO pool) across three different production platforms: 1) using lean production media, 2) using rich production media, and 3) using rich production media in an intensified process. The TKO clones exhibited better bioprocess outcomes showing higher titer and relative specific productivity as compared to controls (FIGS. 6B and 6C, and Table 2), while maintaining comparable product quality attributes (Table 3) across all production platforms. Similar production platforms testing PERK/Bax/Bak TKO pools and clones clearly reveal that deletion of the PERK gene results in higher specific productivity CHO cells expressing antibody (mAb3) or Fab (Fab 1) (FIGS. 7A, 7B and Table 4). These data suggest that the observed increase in specific productivity of Bax/Bak/F'ERK TKO CHO cells is not clone or product specific but is rather a general phenomenon.

TABLE 2
Bioprocess outcomes for mAb3-expressing CHO-K1
TKO cells across different bioprocesses.

| | | Titer | | Relative | | Relative | | % | |
|---|---|---|---|---|---|---|---|---|---|
| | Parameter | (g/L) | | Qp | | IVCC | | Viability | |
| | Day | 10 | 14 | 10 | 14 | 10 | 14 | 10 | 14 |
| Lean | WT | 0.86 | 1.16 | 1.00 | 0.84 | 1.00 | 1.60 | 98.0 | 5.4 |
| Prod. | PERK KO 1 | 0.71 | 0.72 | 0.85 | 0.59 | 0.97 | 1.44 | 88.5 | 1.1 |
| Media | PERK KO 2 | 0.66 | 0.69 | 0.80 | 0.55 | 0.96 | 1.48 | 97.8 | 1.1 |
| | BB DKO | 0.67 | 1.10 | 1.04 | 0.86 | 0.75 | 1.49 | 99.0 | 97.0 |
| | TKO 3 | 1.42 | 2.59 | 3.14 | 2.82 | 0.53 | 1.07 | 96.5 | 94.1 |
| | TKO 2 | 1.55 | 2.62 | 3.45 | 2.86 | 0.52 | 1.07 | 97.4 | 94.4 |
| | TKO 3 | 1.52 | 2.50 | 2.96 | 2.43 | 0.60 | 1.20 | 97.3 | 93.9 |
| | TKO 4 | 1.36 | 2.54 | 2.87 | 2.64 | 0.55 | 1.12 | 96.1 | 94.2 |
| Rich | WT | 1.72 | 1.85 | 1.00 | 0.85 | 1.00 | 1.27 | 97.7 | 0.0 |
| Prod. | PERK KO 1 | 1.31 | 1.15 | 0.86 | 0.60 | 0.89 | 1.12 | 94.2 | 0.0 |
| Media | PERK KO 2 | 1.18 | 1.23 | 0.80 | 0.66 | 0.85 | 1.09 | 96.6 | 0.0 |
| | BB DKO | 1.74 | 2.48 | 1.06 | 0.91 | 0.95 | 1.58 | 98.7 | 96.1 |
| | TKO 1 | 3.66 | 5.30 | 2.68 | 2.32 | 0.79 | 1.33 | 92.9 | 91.7 |
| | TKO 2 | 3.78 | 5.20 | 3.12 | 2.60 | 0.70 | 1.16 | 92.7 | 89.4 |
| | TKO 3 | 4.30 | 6.48 | 3.14 | 2.81 | 0.79 | 1.34 | 95.4 | 90.6 |
| | TKO 4 | 3.66 | 5.32 | 2.36 | 2.06 | 0.90 | 1.50 | 95.3 | 91.3 |
| Rich | WT | 3.76 | 3.82 | 1.00 | 0.87 | 1.00 | 1.17 | 94.0 | 63.7 |
| Prod. | PERK KO 1 | 1.86 | 3.94 | 0.53 | 0.46 | 0.94 | 1.13 | 95.0 | 77.9 |
| Media | PERK KO 2 | 1.84 | 1.92 | 0.49 | 0.41 | 1.01 | 1.23 | 97.0 | 85.3 |
| Intensified | BB DKO | 4.16 | 5.05 | 1.09 | 1.07 | 1.01 | 1.25 | 98.6 | 96.0 |
| | TKO 1 | 7.44 | 8.38 | 2.24 | 2.30 | 0.88 | 1.06 | 97.5 | 89.9 |
| | TKO 2 | 7.36 | 8.28 | 2.35 | 2.29 | 0.83 | 0.96 | 92.0 | 55.8 |

TABLE 3
Product quality of mAb3-expressing CHO-K1 TKO cells across different bioprocesses.

| | | Aggregate | % Charge Variants | | | | | Aggregate | % Charge Variants | | |
|---|---|---|---|---|---|---|---|---|---|---|---|
| | Parameter | % HMWS | Acidic | Main | Basic | | Parameter | % HMWS | Acidic | Main | Basic |
| Lean | WT | 4.39 | 32.12 | 61.02 | 6.86 | Rich | WT | 3.04 | 39.35 | 52.39 | 8.26 |
| Prod. | PERK KO 1 | 3.83 | 36.47 | 57.19 | 0.35 | Prod. | PERK KO 1 | 2.70 | 38.70 | 54.92 | 6.39 |
| Media | PERK KO 2 | 3.31 | 34.80 | 57.76 | 7.43 | Media | PERK KO 2 | 2.89 | 38.54 | 56.11 | 5.35 |
| | BB DKO | | | | | | BB DKO | | | | |
| | Pool | 19.69 | 33.24 | 60.22 | 6.53 | | Pool | 14.19 | 38.71 | 55.09 | 6.21 |
| | TKO 1 | 10.16 | 32.12 | 62.46 | 5.41 | | TKO 1 | 12.03 | 41.65 | 53.82 | 4.53 |
| | TKO 2 | 13.71 | 33.25 | 61.64 | 5.11 | | TKO 2 | 13.16 | 42.73 | 52.52 | 4.76 |
| | TKO 3 | 16.24 | 32.87 | 61.57 | 5.57 | | TKO 3 | 15.70 | 43.82 | 51.83 | 4.35 |
| | TKO 4 | 11.54 | 30.98 | 63.54 | 5.48 | | TKO 4 | 11.90 | 40.77 | 53.48 | 5.75 |

TABLE 4
Bioprocess outcomes for single cell clones of Bax/Bak DKO and PERK/Bax/Bak TKO.

| | mAb3 CHO-K1 Production - TKO Single Cell Clones | | | | | mAb3 CHO-K1 Production - DKO Single Cell Clones | | | |
|---|---|---|---|---|---|---|---|---|---|
| | Day 14 | Rel. | Rel. | Rel. | | Day 14 | Rel. | Rel. | Rel. |
| | Parameter | Titer | Qp | IVCC | | Parameter | Titer | Qp | IVCC |
| Rich | WT | 1.00 | 1.00 | 1.00 | Rich | WT Average | 1.00 | 1.00 | 1.00 |
| Prod. | DKO Pool | 1.34 | 1.08 | 1.24 | Prod. | DKO Average | 1.21 | 1.03 | 1.15 |
| Media | TKO Average | 3.02 | 2.89 | 1.05 | Media | WT1 | 0.65 | 0.54 | 1.16 |
| | TKO 1 | 2.87 | 2.74 | 1.05 | | WT2 | 1.23 | 1.34 | 0.88 |
| | TKO 2 | 2.82 | 3.08 | 0.92 | | WT3 | 1.12 | 1.12 | 0.96 |
| | TKO 3 | 3.51 | 3.32 | 1.06 | | BB DKO1 | 1.34 | 0.82 | 1.58 |
| | TKO 4 | 2.88 | 2.43 | 1.19 | | BB DKO2 | 1.04 | 0.94 | 1.06 |
| | | | | | | BB DKO3 | 1.16 | 1.21 | 0.92 |
| | | | | | | BB DKO4 | 1.01 | 0.99 | 0.97 |
| | | | | | | BB DKO5 | 1.52 | 1.20 | 1.22 |

Western blot analysis revealed that PERK/Bax/Bak TKO clones had higher intracellular levels of antibody heavy chain and light chain in the seed train (FIG. 6A) and production media (FIG. 6D3) relative to the parental line. Additionally, the TKO clones displayed more stabilized PDGFRa expression and no caspase-3 cleavage, indicative of inhibition of apoptosis pathway, in production compared to the parental line (FIG. 6D3). Interestingly, PERK/Bax/Bak TKO clones had higher levels of IRE 1a, a, phosphor-IRE1α and significantly higher levels of spliced XBP-1 transcription factor, indicating that these cells are experiencing increased protein translation and proteostatic stress in production (FIG. 6D). TKO clones also displayed higher levels of Sod2 protein, implying activation of reactive oxygen species (ROS) pathway (FIG. 6D). These findings suggest that activation of PERK branch of the UPR during production cumulatively reduces proteostatic stress by reducing protein translation and attenuation of IRE1a and ROS pathways, thereby mitigating apoptotic cell death in production culture. Further investigation of these production processes linked the increase in specific productivity to increased mRNA levels of the heavy chain and light chain transcripts in the TKO clones compared to the parental cell line (FIG. 6E). This suggests that the PERK branch of the UPR either directly or indirectly, through attenuation of IRE1a or PDGFRa signaling, reduces transgene transcription from the CMV promoter during production.

As mentioned above, to prevent apoptosis due to increased specific productivity in the PERK KO cell lines, PERK was knocked out in an antibody expressing Bax/Bak double knockout cell line (FIG. 6A). Excitingly, a synergistic effect in the TKO clones during production resulted in higher overall titers (up to 8 g/L) and relative specific productivities compared to the parental line while having comparable IVCC and viability (FIGS. 6B, 6C and Table 2). This synergistic effect may be explained by increased IRE1a signaling caused by the deletion of PERK, which has been shown to attenuate IRE1a branch of UPR (Chang, et al., 2018), and in conjunction with a prolonged IRE1 an activity due to attenuation of apoptosis signaling pathway because of deletion of Bax and Bak genes. It was observed increased and prolonged IRE1 a signaling in our TKO clones during production indicated by more phosphorylated IRE1a and increased presence of its downstream target, spliced XBP-1 (FIG. 6D). XBP-1 has been shown to improve bioprocess outcomes transiently (Rajendra, Hougland, Schmitt, & Barnard, 2015) and the observed increase in antibody transcription levels (FIG. 6E) suggests that either activation of PERK branch of the UPR attenuates transgene(s) transcription from the CMV promoter, or PDGFRa and/or IRE1a signaling play a role in enhancing transcription from CMV promoter either directly or through their downstream targets. The exact mechanisms and interplay between these signaling pathways, however, remain to be determined.

The findings presented in the present disclosure suggest that chronic activation of UPR in antibody-expressing CHO cells can trigger poor growth, primarily through the PERK pathway which downregulates PDGFRa levels. The UPR in these cells is largely caused by proteostatic stress in the ER, which can be triggered by many different factors ranging from cell culture parameters to the amino acid sequence and composition of expressed proteins. It is suspected that this is a way to promote adaptive growth when protein production, and hence burden on the ER, increases. Slowing down cellular proliferation and metabolism by regulating PDGFRa levels can allow more time for ER expansion, which is also regulated by the PERK pathway. Knocking out the PERK pathway might allow the cells to grow, but can also result in apoptosis as cells are unable to accommodate the additional stress imposed by high rates of specific productivity and protein synthesis. To bypass this, knocking out the PERK pathway in conjunction with the deletion of components of the apoptotic pathway (Bax/Bak genes) achieves both high rates of specific productivity and increased cell viability. Hence, it is proposed in the present disclosure that knocking out PERK in a mammalian protein expression host cell line with attenuated apoptosis pathway(s) may significantly increase specific productivity and hence culture titers.

The contents of all figures and all references, patents and published patent applications and Accession numbers cited throughout this application are expressly incorporated herein by reference.

1. A modified cell, wherein the cell is modified to reduce or eliminate the expression of two or more endogenous proteins relative to the expression of the endogenous proteins in an unmodified cell, wherein:
(a) one or more of the endogenous proteins having reduced or eliminated expression promotes apoptosis of the modified cell during cell culture; and
(b) one or more of the endogenous proteins having reduced or eliminated expression regulates the unfolded protein response (UPR).

2. The modified cell of claim 1, wherein:
(i) the one or more endogenous proteins that promote apoptosis is selected from the group consisting of: BCL2 Associated X, Apoptosis Regulator (BAX); and BCL2 Antagonist/Killer 1 (BAK); and
(ii) one of the endogenous proteins that regulate the UPR is Protein Kinase R-like ER Kinase (PERK).

3. The modified cell of claim 2, wherein the expression of BAX, BAK, and PERK is reduced or eliminated.

4. The modified cell of claim 1, wherein:
(i) the modified cell is engineered to express a recombinant product of interest; or
(ii) the modified cell is generated from a recombinant cell that expresses a recombinant product of interest.

5. (canceled)

6. The modified cell of claim 4, wherein the one or more endogenous proteins have no detectable expression.

7. The modified cell of claim 4, wherein the recombinant product of interest comprises a viral vector, a viral particle, or a recombinant protein.

8.-9. (canceled)

10. The modified cell of claim 4, wherein the recombinant product of interest is an antibody, an antibody-fusion protein, or an antigen-binding fragment thereof.

11. The modified cell of claim 10, wherein the antibody:
(i) is a multispecific antibody or an antigen-binding fragment thereof; or
(ii) consists of a single heavy chain sequence and a single light chain sequence or antigen-binding fragments thereof.

12. (canceled)

13. The modified cell of claim 10, wherein the antibody is a chimeric antibody, a human antibody, or a humanized antibody.

14. (canceled)

15. The modified cell of claim 4, wherein the recombinant product of interest is encoded by an exogenous nucleic acid sequence integrated in the cellular genome at one or more targeted locations.

16. The modified cell of claim 1, wherein the modified cell:
(i) does not express detectable BAX, BAK, and PERK; or
(ii) the modified cell expresses decreased levels of BAX, BAK, and PERK.

17.-18. (canceled)

19. The modified cell of claim 1, wherein the modified cell is selected from the group consisting of Sf9, CHO, HEK 293, HEK-293T, BHK, A549 and HeLa cell.

20. A composition comprising a modified cell, wherein the cell is modified to reduce or eliminate the expression of two or more endogenous proteins relative to the expression of the endogenous proteins in an unmodified cell, wherein:
(a) one or more of the endogenous proteins having reduced or eliminated expression promotes apoptosis of the modified cell during cell culture; and
(b) one or more of the endogenous proteins having reduced or eliminated expression regulates the unfolded protein response (UPR).

21. A method of producing a recombinant product of interest comprising:
(a) culturing a modified cell, wherein the cell is modified to reduce or eliminate the expression of two or more endogenous proteins relative to the expression of the endogenous proteins in an unmodified cell, wherein:
(i) one or more of the endogenous proteins having reduced or eliminated expression promotes apoptosis of the modified cell during cell culture; and
(ii) one or more of the endogenous proteins having reduced or eliminated expression regulates the unfolded protein response (UPR); and
(b) recovering the recombinant product of interest from a cultivation medium or the modified cells, wherein the modified cells expressing the recombinant product of interest exhibit reduced or eliminated expression of BAX, BAK, and PERK.

22. A method for producing a modified cell, comprising:
(a) applying a nuclease-assisted and/or nucleic acid targeting BAX, BAK, and PERK, in the cell to reduce or eliminate the expression of said endogenous genes, and
(b) selecting the modified cell wherein the expression of said endogenous genes have been reduced or eliminated as compared to an unmodified cell.

23. The method of claim 22, wherein the modification is performed before the introduction of the exogenous nucleic acid encoding the recombinant product of interest, or after the introduction of the exogenous nucleic acid encoding the recombinant product of interest.

24.-27. (canceled)

28. The method of claim 21, wherein:
(i) the recombinant product of interest expressed by the modified cell is encoded by a nucleic acid sequence, wherein the nucleic acid sequence is integrated in the cellular genome of the modified cells at one or more targeted locations; or
(ii) the recombinant product of interest expressed by the modified cells is encoded by a nucleic acid sequence that is randomly integrated in the cellular genome of the modified cells.

29. (canceled)

30. The method of claim 21, wherein the recombinant product of interest comprises a viral vector, a viral particle, or a recombinant protein.

31.-32. (canceled)

33. The method of claim 21, wherein the recombinant product of interest is an antibody, or an antibody-fusion protein, or an antigen-binding fragment thereof.

34. The method of claim 33, wherein antibody:
(i) is a multispecific antibody or an antigen-binding fragment thereof; or
(ii) consists of a single heavy chain sequence and a single light chain sequence or antigen-binding fragments thereof.

35. (canceled)

36. The method of claim 33, wherein the antibody is a chimeric antibody, a human antibody, or a humanized antibody.

37. (canceled)

38. The method of claim 21, further comprising purifying the product of interest, harvesting the product of interest, and/or formulating the product of interest.

39. (canceled)

40. The method of claim 21, wherein the modified cell is selected from the group consisting of Sf9, CHO, HEK 293, HEK 293T, BHK, A549, and HeLa cell.

41. The modified cell of claim 1, wherein the modified cell has a higher specific productivity than a corresponding isolated cell that comprises the polynucleotide and functional copies of each of the wild type Bax, Bak, and PERK genes.

42.-43. (canceled)
